(19) United States
(12) Patent Application Publication
(10) Pub. No.: US 2025/0017252 A1
(43) Pub. Date: Jan. 16, 2025

(54) Fatty acids for boostering ketogenic diet

(71) Applicant: N.V. Nutricia, Zoetermeer (NL)

(72) Inventors: Jose Pablo Silva, Utrecht (NL); Hester Meeusen, Utrecht (NL); Lotte Hendrika Johanna Dopheide, Utrecht (NL); Sebastian Tims, Utrecht (NL)

(73) Assignee: N.V. Nutricia, Zoetermeer (NL)

(21) Appl. No.: 18/817,331

(22) Filed: Aug. 28, 2024

Related U.S. Application Data
(63) Continuation of application No. PCT/EP2023/055212, filed on Mar. 1, 2023.

(30) Foreign Application Priority Data
Mar. 2, 2022 (EP) 22159661.2

(51) Int. Cl. A23L 33/12; A23L 33/15; A23L 33/21

(52) U.S. Cl. CPC A23L 33/12; A23L 33/15; A23L 33/21

(57) ABSTRACT

The invention relates to at least one of C4 or C6 fatty acids and optionally C2 fatty acids for enhancing ketosis associated with a ketogenic diet and/or preventing and/or treating resistance to a ketogenic diet, in a subject adhering to a ketogenic diet, wherein the at least one of C4 or C6 fatty acids and optionally C2 fatty acids are comprised in a non-ketogenic ketogenic diet-booster composition, or the at least C4 or C6 fatty acids and optionally C2 fatty acids are provided in non-ketogenic amounts by a dietary fibre blend comprised in a non-ketogenic ketogenic diet-booster composition.

### FIELD OF THE INVENTION

This invention relates to the use of a non-ketogenic ketogenic diet-booster composition to produce elevated and sustained levels of ketone bodies in the blood, for transitioning into or maintaining nutritional ketosis, and to non-ketogenic ketogenic-diet booster compositions to be used to that end.

### BACKGROUND OF THE INVENTION

Ketosis is the state of elevated blood ketone levels resulting from ketogenic diets, caloric restriction, (therapeutic) fasting and/or supplementation with ketogenic precursors. Ketone bodies represent energy substrates for both peripheral tissues and the central nervous system. The two most abundant and physiologically significant ketone bodies are acetoacetate and β-hydroxybutyrate, while the third ketone body, acetone, is produced as a by-product that the lungs breathe off.

The metabolism of ketone bodies is associated with therapeutic effects in a wide range of therapeutic areas including neurological diseases such as epilepsy and ageing related conditions such as Alzheimer's disease, neuromuscular diseases such as amyotrophic lateral sclerosis, stroke and neurotrauma and metabolic conditions such as obesity, insulin resistance, type-2 diabetes mellitus, Glucose transporter type 1 (Glut1) deficiency, Pyruvate dehydrogenase (PDH) deficiency, glycogen storage diseases and mitochondrial diseases

Amongst others, elevation of ketone bodies in blood have been shown to improve glycaemic control, to suppress inflammation, to decrease mitochondrial reactive oxygen species (ROS) production and favourably alter mitochondrial bioenergetics. Ketone bodies increase the hydraulic efficiency of the heart, simultaneously decreasing oxygen consumption while increasing ATP production. Thus, elevated ketone bodies increase metabolic efficiency and as a consequence, reduce superoxide production and increase reduced glutathione, which in turn increases ATP production and metabolic efficiency further. From a neurological perspective, one of the mechanisms by which ketone bodies achieve their potential is through the biochemical process of their energy metabolism. During cellular respiration in the mitochondria, glucose enters the citric acid cycle via the glycolytic pathway, which in many brain diseases or trauma situations is interrupted or impaired. Ketone bodies enter the citric acid cycle independent from glycolysis and are thereby thought to rescue the energy metabolism of the cell.

The ketogenic diet (KD) is thought to result in adaptive changes to brain energy metabolism that increase the energy production. This is believed to help the neurons to remain stable in the face of increased energy demand and may confer a neuroprotective effect. The classic KD consisting of high fat, low protein and low carbohydrate, typically with ratios of up to 4:1 fats to proteins and carbohydrates, was first introduced in the 1920s for use in human childhood epilepsy. The use of KD was initially suggested in order to mimic the metabolic state and biochemical changes associated with fasting, since fasting was proven to possess anticonvulsant properties.

In subjects on a KD, fatty acids are used as the major source of fuel. These are used through fatty acid oxidation in the cell's mitochondria. The brain is during daytime fueled by glucose, lipids in the form of plasma lipoproteins as such do not cross the blood-brain barrier to a substantial degree. The liver can use long-chain fatty acids though to synthesise the three ketone bodies β-hydroxybutyrate, acetoacetate and acetone. These ketone bodies can cross the blood-brain barrier and act as a glucose substitute during sleep at night or other periods of fasting. Brain neurons have a very limited capacity to use fatty acids as a fuel due to the absent expression of enzymes of the mitochondrial b-oxidation pathway and are therefore dependent on the provision of ketones as a fuel during fasting. When fat and carbohydrates are simultaneously present to neurons, glucose will by far be the preferred energy source while lipids are preferably stored and not used for ketone production. That is why the classical ketogenic diet has a large excess of lipids and hardly any digestible carbohydrates. Typically, the degree of ketogenicity decreases considerably when the amount of digestible carbohydrates in the diet is increased.

The Medium Chain Triglyceride Ketogenic Diet (MCTKD) is a variant of the classic ketogenic diet with about 30-70% MCT fat. The idea of the MCTKD is that because MCTs produce more ketones than regular fat (which consists of mostly LCTs), incorporating high amounts of MCTs into the diet allows patients to stay in ketosis while consuming relatively higher amounts of protein and carbohydrates. The original MCT diet developed in the 1970s derived 60% of its calories from MCT oil. Consuming such a quantity of MCT oil causes abdominal cramps, diarrhoea and vomiting though, which is a severe drawback for its use and generally the MCT content of the MCTKD is limited to be only about 20% of the total calories of the diet. The diet is, like the ketogenic diet, unpalatable which makes it difficult to adhere with. In addition, the health consequences of a very high fat diet for life may be considerable.

The Modified Atkins Diet (MAD) is another, more liberal variation of the traditional ketogenic diet mainly limited in the amount of digestible carbohydrates. While the ketogenic diet is typically given at 4:1 or 3:1 ketogenic ratio, which is the ratio of the amount of fat to the combined amounts of protein and digestible carbohydrates, MAD involves about a 1:1 ratio, which allows for more protein and carbohydrate intake, and requires less fat intake. While the MAD allows for more food choices and is easier to comply with than a classic ketogenic diet, it is less ketogenic and thus less effective than the classic ketogenic diet.

The Low Glycemic Index Diet is another diet variant allowing to induce ketosis. The glycemic index classifies carbohydrates by their potential to raise blood glucose on a scale from 0-100. The low glycemic index diet allows for a greater intake of digestible carbohydrates, but the carbohydrates are limited to those with a low glycemic index (typically considered a glycemic index of 55 or less). In practice low GI nourishment induces a mild ketogenic state and patients find the diet easier to adhere to than to a classic ketogenic diet.

Though a KD is effective at raising blood ketone levels and has potential broad applications, patient compliance is low due to the restrictive nature of the diet. Additionally, some epilepsy patients develop a resistance to ketosis-induction or experience an increase in seizure frequency or severity despite adhering to the KD. Also subjects on a ketogenic diet experience an immediate shift back to glucose utilization, meaning a decrease in ketone production and levels, if carbohydrates are consumed over the restrictive amount. Accordingly, there remains a need to develop compositions and methods that do not suffer from the drawbacks of existing ketogenic diets. Such compositions and methods would be useful to improve the compliance of subjects using a ketogenic diet and/or enhance ketosis state and/or increase the production of ketone bodies.

### SUMMARY OF THE INVENTION

The inventors have observed that after administration of a non-ketogenic ketogenic diet-booster composition the level of ketone bodies produced from a ketogenic diet is enhanced. The inventors found that at least one of C4 or C6 fatty acids and in addition optionally C2 fatty acids is beneficial for therapeutic use in (i) enhancing ketosis and/or (ii) preventing and/or treating resistance to a ketogenic diet, in a subject adhering to/taking a ketogenic diet, wherein the at least one of C4 or C6 fatty acids and optionally C2 fatty acids or derivatives thereof are comprised in a non-ketogenic ketogenic diet-booster composition, or the at least one of C4 or C6 fatty acids and optionally C2 fatty acids are provided in non-ketogenic amounts by a dietary fibre blend comprised in a non-ketogenic ketogenic diet-booster composition. Treating resistance to a ketogenic diet includes reducing the severity of resistance to a ketogenic diet.

Worded differently, the invention pertains to the use of a non-ketogenic ketogenic diet-booster composition in the manufacture of a product for (i) enhancing ketosis and/or (ii) preventing and/or treating resistance to a ketogenic diet, wherein the at least one of C4 or C6 fatty acids and optionally C2 fatty acids are comprised in a non-ketogenic ketogenic diet-booster composition, or the at least one of C4 or C6 fatty acids and optionally C2 fatty acids are provided in non-ketogenic amounts by a dietary fibre blend comprised in a non-ketogenic ketogenic diet-booster composition.

Also, the invention pertains to a (non-therapeutic) method for (i) enhancing ketosis and/or (ii) preventing/treating resistance in a (healthy) subject in need thereof and adhering to a ketogenic diet, comprising administering to the subject at least one of C4 or C6 fatty acids and optionally C2 fatty acids, wherein the at least one of C4 or C6 fatty acids and optionally C2 fatty acids are comprised in a non-ketogenic, ketogenic diet-booster composition, or the at least one of C4 or C6 fatty acids and optionally C2 fatty acids are provided in non-ketogenic amounts by a fibre blend in a non-ketogenic, ketogenic diet-booster composition.

In particular, the present invention provides non-ketogenic ketogenic diet-booster compositions, methods of use and use to increases the ketone production of subjects on a ketogenic diet, sustain and/or improve ketosis and/or to prevent or treat tolerance to a ketogenic diet in subjects on a ketogenic diet. In a particular embodiment the ketogenic booster composition increases the amount of ketone bodies produced from a ketogenic diet, meaning less dietary fat is needed to sustain ketosis state in a subject on a ketogenic diet.

In a particular embodiment the non-ketogenic ketogenic diet-booster composition is for use in the prevention of seizures in a subject with epilepsy on a ketogenic diet.

The present invention provides for a non-ketogenic ketogenic diet-booster composition comprising
(i) at least one of C4 and C6 fatty acids, more preferably at least C4 fatty acids
(ii) optionally one or more of C2 fatty acids, citric acid and/or nicotinamide riboside.

The invention further pertains to a non-ketogenic ketogenic diet-booster composition essentially consisting of
(i) 0.2 mg to 1.2 g C4 fatty acids;
(ii) 5 mg to 17 g C6 fatty acids;
(iii) 10 mg to 20 g citrate; and
(iv) 0.5 mg to 1.5 g nicotinamide riboside,
wherein the amounts are per serving based on the dry weight of the composition.

The invention further pertains to a non-ketogenic ketogenic diet-booster composition essentially consisting of
(i) 20 mg to 1.2 g C2 fatty acids;
(ii) 4 mg to 480 mg C4 fatty acids;
(iii) 260 mg to 3.3 g C6 fatty acids;
(iv) 0.2 mg to 4.4 g citrate; and
(v) 12 mg to 286 mg nicotinamide riboside,
wherein the weight ratio of C2 and C4 fatty acids:C6 fatty acids is between 1:1.8 and 1:2.1, preferably between 1:1.9 and 1:2.0;
the weight ratio of C2 and C4 fatty acids:citrate is between 1:2.4 and 1:2.7, preferably between 1:2.5 and 1:2.6 and
the weight ratio of C2 and C4 fatty acids:nicotinamide riboside is between 1:0.15 and 1:0.19, preferably between 1:0.16 and 1:0.18 based on dry weight and wherein the total weight of the composition is between about 10-20 grams.

### LIST OF FIGURES

The present invention will be discussed in more detail below, with reference to the attached figures.

FIGS. 1A-1D: Illustrate the effect of C2 fatty acids on ketone production by liver cells.

FIGS. 2A-2D: Illustrate the effect of C4 fatty acids on ketone production by liver cells.

FIGS. 3A-3D: Illustrate the effect of C6 fatty acids on ketone production by liver cells.

FIGS. 4A-4D: Illustrate the effect of citric acid on ketone production by liver cells.

FIGS. 5A-5C: Illustrate the effect of nicotinamide riboside on ketone production by liver cells.

FIGS. 6A-6C: Illustrate the effect of nicotinamide riboside under ketogenic conditions on ketone production by liver cells.

FIGS. 7A and 7B: Illustrate the effect of a combination of C4 and C6 fatty acids on ketone production by liver cells.

FIG. 8: Illustrates the effect of a combination of C4, C6, citrate and either NR or NAM on ketone production by liver cells.

FIG. 9: Illustrates butyrate to propionate weight ratio of 24-hour fermentation of dietary fibers.

### LIST OF EMBODIMENTS

1. At least one of C4 or C6 fatty acids and optionally C2 fatty acids for therapeutic use in
(i) enhancing ketosis associated with a ketogenic diet and/or
(ii) preventing and/or treating resistance to a ketogenic diet,

In a subject adhering to a ketogenic diet, wherein the at least one of C4 or C6 fatty acids and optionally C2 fatty acids are comprised in a non-ketogenic ketogenic diet-booster composition, or the at least C4 or C6 fatty acids and optionally C2 fatty acids are provided in non-ketogenic amounts by a butyrogenic dietary fiber or butyrogenic dietary fiber blend comprised in a non-ketogenic ketogenic diet-booster composition.

2. The at least one of C4 or C6 fatty acids and optionally C2 fatty acids for use according to embodiment 1 wherein enhancing ketosis means increasing blood ketone concentrations in a subject on a ketogenic diet using the non-ketogenic ketogenic diet-booster composition in comparison to the blood ketone concentration in a subject consuming the same ketogenic diet and not using the non-ketogenic ketogenic diet-booster composition.

3. The at least one of C4 or C6 fatty acids and optionally C2 fatty acids for use according to embodiments 1 and 2 wherein the non-ketogenic ketogenic diet-booster composition further comprises one or more compounds selected from a) citric acid and/or b) nicotinamide riboside and/or nicotinamide.

4. The at least one of C4 or C6 fatty acids and optionally C2 fatty acids for use according to any one of the previous embodiments wherein the non-ketogenic ketogenic diet-booster composition is in the form of a nutritional supplement.

5. The at least one of C4 or C6 fatty acids and optionally C2 fatty acids for use according to any one of the previous embodiments,
wherein the at least C4 or C6 fatty acids and optionally C2 fatty acids are provided in non-ketogenic amounts by a butyrogenic dietary fibre or butyrogenic dietary fiber blend, and
wherein the dietary fibers are selected from pectins, mucilages, gums, galacto-oligosaccharides, oligofructan, inulin, polyfructoses, fructo-oligosaccharides, arabinoglactans, hemicelullose, resistant starch, fuji soy, oligosaccharides, or mixtures of thereof, and
wherein the dietary fibers have a fermentation profile providing butyrate and propionate in a weight ratio of at least 1, preferably at least 1.1.

6. The at least one of C4 or C6 fatty acids and optionally C2 fatty acids thereof for use according to any one of the previous embodiments wherein the amount of C4 fatty acids is 0.2 mg to 12 mg per kilogram bodyweight per day and/or the amount of C6 fatty acids is 5 mg to 185 mg per kilogram bodyweight per day.

7. The at least one of C4 or C6 fatty acids and optionally C2 fatty acids for use according to any one of the previous embodiments wherein the non-ketogenic ketogenic diet-booster composition is administered one to six times per day, preferably three to four times per day, or continuously, or wherein the non-ketogenic ketogenic diet-booster composition is administered continuously through a tube.

8. The at least one of C4 or C6 fatty acids and optionally C2 fatty acids for use according to any one of the previous embodiments wherein the subject adhering to a ketogenic diet is suffering from a condition selected from the group of conditions consisting of
i) neurological diseases including epilepsy, stroke, traumatic brain injury, migraine;
ii) neurodegenerative diseases including Alzheimer's disease, Parkinson's disease and age-related mild cognitive impairment (MCI);
iii) neuromuscular diseases including amyotrophic lateral sclerosis, ageing-induced muscle inactivation, muscle wasting and sarcopenia, or elderly at risk of developing or suffering from frailty;
iv) psychiatric diseases including attention deficit hyperactivity disorder (ADHD) and autism spectrum disorders;
v) metabolic conditions including insulin resistance, type-1 and type-2 diabetes mellitus, Glucose transporter type 1 (Glut1) deficiency, Pyruvate dehydrogenase (PDH) deficiency, glycogen storage diseases and mitochondrial diseases;
vi) oncological disorders;
vii) cardiovascular diseases leading to heart failure.

9. The at least one of C4 or C6 fatty acids and optionally C2 fatty acids for use according to any one of the previous embodiments wherein the subject adhering to a ketogenic diet is a subject suffering from epilepsy.

10. The at least one of C4 or C6 fatty acids and optionally C2 fatty acids for use according to any one of the previous embodiments wherein top-level ketosis is increased.

11. The at least one of C4 or C6 fatty acids and optionally C2 fatty acids for use according to any one of the previous embodiments wherein the non-ketogenic ketogenic diet-booster composition comprises
(i) C4 fatty acids
(ii) C6 fatty acids
(iii) citric acid, and
(iv) nicotinamide riboside or nicotinamide, and optionally
(v) C2 fatty acids.

12. The at least one of C4 or C6 fatty acids and optionally C2 fatty acids for use according to any one of the previous embodiments wherein the non-ketogenic ketogenic diet-booster composition is administered at a dose of
(i) 0.2 mg to 12 mg per kilogram bodyweight per day C4 fatty acids
(ii) 6.5 mg to 163 mg per kilogram bodyweight per day C6 fatty acids
(iii) 0.011 gram to 0.39 gram per kilogram bodyweight per day citric acid, and
(iv) 0.6 mg to 14.3 mg per kilogram bodyweight per day nicotinamide riboside or equivalent dose range of nicotinamide, and optionally
(v) 0.5 mg to 30 mg per kilogram bodyweight per day C2 fatty acids.

13. Non-therapeutic method for
(i) enhancing ketosis and/or
(ii) preventing resistance to a ketogenic diet
in a healthy subject adhering to a ketogenic diet, comprising administering to the subject at least one of C4 or C6 fatty acids and optionally C2 fatty acids, wherein the one of C4 or C6 fatty acids and optionally C2 fatty acids are comprised in a non-ketogenic, ketogenic diet-booster composition, or the at least C2 or C4 fatty acids or derivatives thereof are provided in non-ketogenic amounts by a butyrogenic dietary fiber blend in a non-ketogenic, ketogenic diet-booster composition.

14. Non-therapeutic method according to embodiments 13 wherein the method is for promoting non-therapeutic weight loss and/or improving recovery after exercise and wherein optionally the promotion of non-therapeutic weight loss is in a subject having a BMI between 18 and 25, preferably between 20 and 25.

15. Non ketogenic, ketogenic diet-booster composition essentially consisting of
(i) 0.2 mg to 1.2 g C4 fatty acids;
(ii) 5 mg to 17 g C6 fatty acids;
(iii) 10 mg to 20 g citrate; and
(iv) 0.5 mg to 1.5 g nicotinamide riboside or nicotinamide wherein the amounts are based on the dry weight of the composition and wherein the amounts are per serving and wherein optionally the composition is in the form of a supplement.

16. At least one of C4 or C6 fatty acids and optionally C2 fatty acids for therapeutic use in
(i) enhancing ketosis associated with fasting or intermittent fasting,
in a subject adhering to a fasting or intermittent fasting regimen, wherein the at least one of C4 or C6 fatty acids and optionally C2 fatty acids are comprised in a non-ketogenic ketogenic diet-booster composition, or the at least one of C4 or C6 fatty acids and optionally C2 fatty acids are provided in non-ketogenic amounts by a butyrogenic dietary fiber blend comprised in a non-ketogenic ketogenic diet-booster composition.

### DETAILED DESCRIPTION OF THE INVENTION

The present invention provides for non-ketogenic ketogenic diet-booster compositions, methods of use and use to increases the ketone production of subjects on a ketogenic diet, sustain and/or improve ketosis and/or to prevent or treat tolerance to a ketogenic diet in subjects on a ketogenic diet.

At least one of C4 or C6 fatty acids and optionally C2 fatty acids is found to be beneficial for use in (i) enhancing ketosis and/or (ii) preventing and/or treating resistance to a ketogenic diet, in a subject adhering to/taking a ketogenic diet, wherein the at least one of C4 or C6 fatty acids and optionally C2 fatty acids are comprised in a non-ketogenic ketogenic diet-booster composition, or the at least one of C4 or C6 fatty acids and optionally C2 fatty acids are provided in non-ketogenic amounts by a dietary fibre blend comprised in a non-ketogenic ketogenic diet-booster composition. Optionally the non-ketogenic ketogenic diet-booster further comprises one or more of citric acid and/or nicotinamide riboside. It was found that the at least one of C4 or C6 fatty acids and optionally C2 fatty acids beneficially allows to reduce the amount of calories from a ketogenic diet that a subject adhering to/taking a ketogenic diet is needed to use to sustain and/or improve ketosis.

It was found by the inventors that the use of C4 and/or C6 fatty acids and optionally C2, when used in amounts that give rise to sub-physiological plasma levels, increases or enhances the production of ketones of a ketogenic diet while not being a metabolic substrate or precursor for the production of ketone bodies themselves. It was also observed that C4 and C6 and optionally one or more of citric acid and/or nicotinamide riboside have a synergistic effect on ketosis.

### Definitions

Throughout this application, the following terminology and abbreviations may be used.

Fibres are non-digestible carbohydrates. Non-digestible carbohydrates are carbohydrates that are resistant to digestion and absorption in the human stomach and small intestine and enter the colon intact. So, compounds like lactose, maltose, glucose, standard maltodextrin, and standard starch are regarded as digestible. Fibres can be fermentable in the colon, or non-fermentable. The term “fermentable” refers to the capability to undergo (anaerobic) breakdown by micro-organism in the lower part of the gastro-intestinal tract, e.g., colon, to smaller molecules, in particular short chain fatty acids and lactate. The fermentability may be determined by the method described in Am. J. Clin. Nutr. 53, 1418-1424 (1991).

Butyrogenic dietary fibers are fermentable dietary fibers. Butyrogenic dietary fibers or blends produce more hexanoate and butyrate than propionate and pentanoate SCFA. For the sake of this specification, the butyrogenic dietary fibers for use in the present invention are defined as dietary fibers or dietary fiber blends that demonstrate to produce butyrate and propionate in a beneficial ratio. Upon fermentation of butyrogenic dietary fibers the production of pentanoate and hexanoate is negligible,

A classical ketogenic diet comprises an amount of lipids (by weight), which is typically 4-fold the weight of the sum of proteins and digestible carbohydrates. In the context of the invention, the so-called ketogenic (weight) ratio is the weight ratio of the amount of lipid to the combined weight amounts of protein and digestible carbohydrates in the composition. The ketogenic diet referred to in the context of the invention may be any type of ketogenic diet known in the preferably characterized by a ketogenic weight ratio between 1:1 and 4:1 which is the ratio of the amount of fat to the combined amounts of protein and digestible carbohydrates. Within the aforementioned (sub)ranges of ketogenic ratios of the invention, the ketogenic diet composition used within the context of the invention preferably comprises a lipid content that is at least twice the carbohydrate content by weight. A ketogenic diet composition as used in the context of the invention induces, when adhered thereto, a state of ketosis.

In an alternative embodiment a subject may follow a dietary intervention that allows to induce ketosis in a subject by adhering to an intermittent fasting regimen. Intermittent fasting (IF) as used encompasses a diet or eating pattern with regularly occurring periods of food abstention longer than a typical overnight fast. In contrast to traditional methods of continuous energy restriction, IF programs utilize intermittent energy restriction by interspersing periods of less-restricted or unrestricted feeding with periods of severely limited energy intake. Several forms of IF have been described, including time-restricted feeding (TRF) (restricting food intake to specific time periods of the day, typically between 8 to 12 hours each day), Alternate-day fasting (ADF) (alternating between no calories for one day and eating without restriction the next), alternate-day modified fasting (alternating between few calories one day and eating without restriction the next) and periodic fasting (fasting 1 or 2 days per week and consuming food ad libitum on 5 to 6 days per week)

The term “Ketosis” as used herein refers to a subject having blood ketone levels above 0.5 mmol/L. Ketone levels sustained above 0.5 mmol/L and ideally in the range of 1 to 3 mmol/L offer therapeutic effects in humans [Anderson J C et al. Obes Sci Pract. 2021; 7(5):646-656)]. Levels of ketones in the blood above 10 mmol/L are associated with signs of ketoacidosis. While ketosis refers to a state of elevated ketones, ketoacidosis is a pathological and potentially life-threatening condition amongst others resulting in a decrease in blood pH and may induce a coma.

In the context of the invention a “non-ketogenic ketogenic diet-booster composition” is a composition or combination of compounds which does not increase or enhance the production of ketones by itself (i.e. it is a non-ketogenic composition), but it improves and/or enhances (boosts) the production of the amount of ketones induced by a ketogenic diet, or ketogenic interventions such as intermittent time periods of fasting or carbohydrate restriction. The composition thus increases (‘boosts’) the ketone amount production of subjects on a ketogenic diet and allows to sustain ketosis, and preferably also increases the rate of ketone production without serving as or being a substrate for the production of ketone bodies. The increase in ketone body production induced by the non-ketogenic ketogenic diet-booster composition in a subject consuming a ketogenic diet or ketogenic substrate is an increase with respect to the ketone body production with the same amount of ketogenic diet or ketogenic substrate yet in the absence of the ‘non-ketogenic ketogenic diet-booster composition’. The non-ketogenic ketogenic diet-booster composition is not a metabolic substrate for the production of ketone bodies itself, i.e., the composition is non-ketogenic, but increases or enhances the production of ketone bodies of a ketogenic composition or ketogenic diet. Optionally a compound in the composition may sometimes give rise to minor amounts of ketone bodies that in itself do not increase plasma ketone concentrations and/or cannot lead to ketosis at the specified amounts provided yet stimulates the formation of ketone bodies of a ketogenic composition in a more than additive manner.

The non-ketogenic ketogenic diet-booster composition also does not have a ketogenic ratio of between 1:1 and 4:1. The non-ketogenic ketogenic diet-booster composition may be incorporated within a ketogenic diet or may be a separate composition.

In an alternative embodiment “non-ketogenic ketogenic diet-booster” is a composition or combination of compounds which does not increase or enhance the production of ketones by itself in order to provide for or sustain a state of ketosis (i.e. it is a non-ketogenic composition), i.e. without serving as or being a substrate for the production of ketone bodies on its own or without increasing the amount of systemic and/or blood ketone body levels at the specified dosing provided. The composition improves/increases the production of the amount of ketones induced by a ketogenic intervention such as intermittent time periods of fasting. The composition increases (‘boosts’) the ketone amount production of subjects following an intermittent fasting regimen and allows to sustain ketosis and may also increase the rate of ketone production.

The expression “increasing ketone body production” means that the amount of systemic and/or blood ketone bodies is higher in an individual on i) a ketogenic diet fed (co-administered) with ii) the non-ketogenic ketogenic diet-booster composition according to the invention, in comparison with the individual being given the same (amount and type) ketogenic diet yet without the non-ketogenic ketogenic diet-booster composition. In one aspect the non-ketogenic ketogenic diet-booster composition decreases the time until therapeutically efficacious ketone concentrations are reached.

A non-ketogenic ketogenic diet-booster composition allows to prevent a loss in ketone body production and maintains ketosis in a subject that is on a long-term ketogenic diet, the non-ketogenic ketogenic diet-booster composition prevents tolerance to the ketogenic diet that is a loss in therapeutic efficacy over time. The non-ketogenic ketogenic diet-booster composition according to the invention does not give rise to ketoacidosis.

In the context of the invention a subject adhering to or taking a ketogenic diet is a mammal, preferably a human. Where reference is made to amounts per kilogram bodyweight reference this is the bodyweight of the subject adhering to or taking a ketogenic diet and using the non-ketogenic ketogenic diet-booster.

The expression “non-ketogenic composition” as used herein refers to a composition that upon administration does not lead to the formation of ketone bodies on its own or cannot increase the amount of systemic and/or blood ketone bodies i.e. a non-ketogenic composition is not a metabolic substrate for the formation of ketone bodies and/or may optionally give rise to the production of minor amounts of ketone bodies that in itself cannot lead to ketosis, yet stimulates the formation of ketone bodies in the presence of a ketogenic composition in a more than additive manner. Non-ketogenic amounts as used herein thus indicate amounts of a compound wherein such a compound in itself does not serve as a substrate for, or lead to the formation of not more than just minimal amounts of ketone bodies unable to induce ketosis by itself. Accordingly, the amount of systemic and/or blood ketone bodies do not rise in reaction thereto.

In contrast, a ketogenic composition is a composition that may be used to promote the formation of ketone bodies and ketosis in a subject, wherein the composition itself comprises ketone bodies and/or substrates that means precursors, usually in the form of fatty acids, for the formation of ketone bodies. A ketogenic diet is an exemplary ketogenic composition.

The non-ketogenic ketogenic diet-booster improves the production of the amount of ketones induced by a ketogenic diet, meaning that it improves the production of the top-level ketosis of the subject adhering to a ketogenic diet.

The term “top-level ketosis” as used herein indicates the maximum amount of blood ketones that a subject on a ketogenic diet may reach considering the maximum amount of ketones produced by hepatocytes when provided a ketogenic fat blend. An increase or enhancement in top-level ketosis in the context of the invention refers to the effects of the ketogenic booster composition as disclosed herein on maximum blood ketone amounts relative to the maximum amount of blood ketones in a subject consuming a similar amount of ketogenic diet without use of the non-ketogenic ketogenic diet-booster composition.

In the context of the invention resistance to a ketogenic diet means a subject on a ketogenic diet suffers from resistance to ketosis-induction meaning blood ketones remain or become too low for therapeutically effective ketosis or means a subject experiences for example an increase in seizure frequency or severity despite adhering to the KD.

The term C2 fatty acid, C4 fatty acid and C6 fatty acid cover any compound that in an animal body is converted into ethanoic acid or ethanoate anion, butyric acid or butanoate anion and caproic acid or caproate anion (hexanoic acid or hexanoate) respectively. Derivatives foreseen herein under the terms C2, C4 and C6 fatty acid include food approved esters, complexes, or salts from the fatty acids. Derivatives of the fatty acids according to the present invention include (partial) glycerol, physiologically acceptable complexes, or salts thereof such as calcium, potassium, sodium, magnesium, ammonium salts and food approved esters.

The term “fatty acyl chain” as used in the present invention refers to a fatty acid linked by an ester bond.

The term “supplement”, or “dietary supplement”, as used herein, refers to a nutritional product that provides nutrients to an individual that may otherwise not conveniently be consumed in sufficient quantities by said individual. Supplements typically provide the selected nutrients while not representing a significant portion of the overall nutritional needs of the subject. Typically, they do not represent more than 0.1%, 1%, 5%, 10% of the daily energy need of the subject.

A glyceride is an ester formed between glycerol and a carboxylic acid, usually in the form of a fatty acid. As an example, a triglyceride (also known as a triacylglycerol) is a triester that is derived from glycerol and three carboxylic acids. Under hydrolysis conditions such as those during digestion, triglycerides may be a source of carboxylic acids or fatty acids. For instance, tributyrin is potentially a source of three moles of butyric acid per mole of tributyrin. Partial glycerides are esters of glycerol with fatty acids, where not all the hydroxyl groups of glycerol are esterified; mono- and di-butyrin are also sources of butyric acid according to the invention providing for one and two moles of butyric acid per mole respectively.

In the context of the in invention, where reference is made to an amount of ethanoic acid, butyric acid or caproic acid respectively, where different sources or derivatives are used the amounts are calculated in terms of the corresponding number of molecules (in mole).

In this document and in its claims, the verb “to comprise” and its conjugations is used in its non-limiting sense to mean that items following the word are included, but items not specifically mentioned are not excluded. “Consisting essentially of” means that the non-ketogenic ketogenic diet-booster composition contains the active ingredients and possibly additional compounds, provided these do not materially affect the essential characteristics of the composition. In addition, reference to an element by the indefinite article “a” or “an” does not exclude the possibility that more than one of the elements is present, unless the context clearly requires that there be one and only one of the elements. The indefinite article “a” or “an” thus usually means “at least one”. Where reference is made to % herein it means wt % unless indicated differently.

### Therapeutic Ketosis

Accordingly, an aspect of the present disclosure is a non-ketogenic ketogenic diet-booster composition comprising at least one of C4 or C6 fatty acids and optionally C2 fatty acids and (ii) optionally one or more citric acid and/or nicotinamide riboside, the composition comprising the combination in an amount effective for improving or sustaining ketosis in a subject on a ketogenic diet in the treatment or prevention or improvement of the symptoms of at least a condition selected from the group of conditions consisting of
i) neurological diseases including epilepsy, stroke, traumatic brain injury, migraine;
ii) neurodegenerative diseases including Alzheimer's disease, Parkinson's disease and age-related mild cognitive impairment (MCI);
iii) neuromuscular diseases including amyotrophic lateral sclerosis, ageing-induced muscle inactivation, muscle wasting and sarcopenia, or elderly at risk of developing or suffering from frailty;
iv) psychiatric diseases including attention deficit hyperactivity disorder (ADHD) and autism spectrum disorders;
v) metabolic conditions including overweight and obesity (i.e. a subject having a BMI above 25 and 30 respectively), insulin resistance, type-1 and type-2 diabetes mellitus, Glucose transporter type 1 (Glut1) deficiency, Pyruvate dehydrogenase (PDH) deficiency, glycogen storage diseases and mitochondrial diseases;
vi) oncological disorders;
vii) cardiovascular diseases leading to heart failure.

In an aspect of the invention the non-ketogenic ketogenic diet-booster composition may increase the ketone production and decrease the time until therapeutically efficacious ketone concentrations are reached. A decrease in the time to reach a state of ketosis is of particular use in refractory status epilepticus.

In a further aspect of the invention the non-ketogenic ketogenic diet-booster composition may also reduce the amount of fat in a ketogenic diet needed to reach and/or maintain a state of ketosis. In an embodiment the amount of fat in the ketogenic diet may be reduced by about 10% on the basis of a same total amount of calories in the ketogenic diet, provided the ketogenic ratio is maintained within the levels of 1:1 and 4:1. By reducing the amount of fat in the ketogenic diet also the ketogenic ratio is reduced by about 10%. It is believed that a reduction in the amount of fats in the ketogenic diet provides for a diet that is easier to comply with, i.e., it improves patient compliance.

The subject is preferably a human subject in therapeutic need of ketosis.

### Non-Therapeutic Ketosis

In another aspect of the invention there is provided a non-therapeutic method for (i) enhancing ketosis and/or (ii) preventing/treating resistance in a healthy subject taking a ketogenic diet, comprising administering to the subject at least one of C4 or C6 fatty acids and optionally C2 fatty acids, wherein the at least one of C4 or C6 fatty acids and optionally C2 fatty acids are comprised in a non-ketogenic, ketogenic diet-booster composition, or the at least one of C4 or C6 fatty acids and optionally C2 fatty acids are provided in non-ketogenic amounts by a fibre blend in a non-ketogenic, ketogenic diet-booster composition. The non-ketogenic, ketogenic diet-booster composition may be administered as a separate composition or be comprised in a ketogenic diet composition. In a particular aspect the healthy subject is taking a ketogenic diet for use in cosmetic weight loss or muscle recovery post exercise, for instance athletes.

In the context of the non-therapeutic method a healthy subject taking a ketogenic diet for use in cosmetic weight loss preferably has a body mass index (BMI) between 25 and 18, more preferably between 25 and 20.

### Ingredients of the Non-Ketogenic Ketogenic Diet-Booster Composition

### Fatty Acids

Preferably the non-ketogenic ketogenic diet-booster composition comprises lipids comprising one or more fatty acids with six carbon atoms or less. Fatty acids according to the invention which are not attached to other molecules are referred to as free fatty acids (FFA). In a preferred embodiment the fatty acids as used in the present composition are unbranched, more preferably unbranched, and even numbered. Most preferably the present non-ketogenic ketogenic diet-booster composition comprises the fatty acids comprising two (acetic acid and/or acetate) and/or four (butyric acid and/or butyrate) and/or six (hexanoic acid and/or hexanoate) carbon atoms. The non-ketogenic ketogenic diet-booster composition preferably comprises at least one of C4 or C6 fatty acids or derivatives thereof, preferably at least C4 fatty acids and optionally C2 fatty acids. In a preferred embodiment preferably at least C4 fatty acids, preferably C4 and C6 fatty acids are present. Preferably the non-ketogenic ketogenic diet-booster composition does not comprise C8 or higher fatty acids.

The fatty acids may be present in the non-ketogenic ketogenic diet-booster composition as such in free fatty acid form or as lipids comprising the fatty acids, preferably as lipids. In the context of the in invention, where reference is made to an amount of fatty acid, where different sources or derivatives are used the amounts are calculated in terms of the corresponding (weight) amount of said fatty acid.

Lipids comprising fatty acids as preferably used in the present invention are preferably selected from the group consisting of triglycerides, diglycerides, monoglycerides, glycolipids, phospholipids and lysophospholipids. In one embodiment the present composition contains triglycerides comprising fatty acyl chains and/or phospholipids comprising fatty acyl chains, more preferably triglycerides comprising fatty acyl chains. The present glyceride preferably has at least one, more preferably at least two fatty acyl chains, even more preferably 3 fatty acyl chains.

Lipids comprising fatty acyl chains are degraded by lingual, gastric, duodenal (i.e. pancreatic) and small intestinal lipases. Hence, administration of lipids comprising fatty acyl chains results in the release of fatty acids in the stomach, the duodenum, in the jejunum and ileum.

A preferred source of lipids containing fatty acyl chains is by chemical synthesis. The source of the lipids may be one or more of animal, plant, fermented, microalgae, GMO, non-GMO or mixtures thereof. An alternative option are lipids obtained from milk from non-human mammals, preferably cow's milk, goat milk, sheep milk, horse milk, buffalo milk, yak milk, reindeer milk, donkey milk and camel milk, particularly cow's milk and/or goat milk. Milk lipid is sometimes also referred to as milk fat or butter fat. In a preferred embodiment, lipids comprising short chain fatty acyl chains are synthesised from glycerol and fatty acids by enzymes such as esterases. Examples are triacetin and tributyrin.

The non-ketogenic ketogenic diet-booster composition preferably comprises at least one of C4 or C6 fatty acids or derivatives thereof. In one embodiment when in addition optionally C2 fatty acids or derivatives thereof are present in the non-ketogenic ketogenic diet-booster composition, the C2 fatty acids or derivatives thereof are present in therapeutically effective dose to increase the ketone production of subjects on a ketogenic diet, sustain and/or improve ketosis and/or to prevent or treat tolerance to a ketogenic diet in subjects on a ketogenic diet.

The ketogenic diet referred to in the context of the invention preferably has a ketogenic weight ratio between 1:1 and 4:1.

C2 fatty acids or derivatives thereof are optionally present in the non-ketogenic ketogenic diet-booster composition in an amount to provide a daily dosage of C2 fatty acids in the range of 0.5 mg to 30 mg per kilogram bodyweight per day, preferably 1.5 mg to 7.5 mg per kilogram bodyweight per day, more preferably 2.0 mg to 3.0 mg per kilogram bodyweight per day.

In one embodiment C2 fatty acids are present in the non-ketogenic ketogenic diet-booster composition as triglyceride form triacetin or glycerol triacetate. Triacetin is, if present, preferably present in the non-ketogenic ketogenic diet-booster composition in an amount to provide a daily dosage of triacetin in the range of 0.6 mg to 36 mg per kilogram bodyweight per day, preferably 3 mg to 9 mg per kilogram bodyweight per day, more preferably 2.4 mg to 3.6 mg per kilogram bodyweight per day.

In one embodiment the non-ketogenic ketogenic diet-booster composition comprises 0.2 mg to 4 g, more preferably 0.5 mg to 3 g C2 short chain fatty acids or derivatives thereof per serving based on dry weight of the composition.

In an embodiment, when C4 fatty acids or derivatives thereof are present in the non-ketogenic ketogenic diet-booster composition, the C4 fatty acids are present in therapeutically effective dose to increases the ketone production of subjects on a ketogenic diet, sustain and/or improve ketosis and/or to prevent or treat tolerance to a ketogenic diet in subjects on a ketogenic diet. In a preferred embodiment preferably at least C4 and optionally C2 fatty acids are present.

C4 fatty acids are preferably present in the non-ketogenic ketogenic diet-booster composition in an amount to provide a daily dosage of C4 fatty acids in the range of 0.2 mg to 12 mg per kilogram bodyweight per day, preferably 1 mg to 6 mg per kilogram bodyweight per day, more preferably 1.5 mg to 3 mg per kilogram bodyweight per day.

In one embodiment C4 fatty acids are present in the non-ketogenic ketogenic diet-booster composition as triglyceride form tributyrin or glycerol tributyrate. Tributyrin is, if present, preferably present in the non-ketogenic ketogenic diet-booster composition in an amount to provide a daily dosage of tributyrin in the range 0.2 mg to 14 mg per kilogram bodyweight per day, preferably 1 mg to 7 mg per kilogram bodyweight per day, more preferably 2 mg to 5 mg per kilogram bodyweight per day.

In one embodiment the non-ketogenic ketogenic diet-booster composition comprises 0.1 mg to 1.5 g, more preferably 0.2 mg to 1.2 g C4 short chain fatty acids or derivatives thereof per serving based on dry weight of the composition.

In an embodiment, when C6 fatty acids are present in the non-ketogenic ketogenic diet-booster composition, the C6 fatty acids are present in therapeutically effective dose to increases the ketone production of subjects on a ketogenic diet, sustain and/or improve ketosis and/or to prevent or treat tolerance to a ketogenic diet in subjects on a ketogenic diet.

C6 fatty acids are preferably present in the non-ketogenic ketogenic diet-booster composition in an amount to provide a daily dosage of C6 fatty acids in the range of 6 mg to 160 mg per kilogram bodyweight per day, preferably 30 mg to 85 mg per kilogram bodyweight per day, more preferably 50 mg to 70 mg per kilogram bodyweight per day.

In one embodiment C6 fatty acids are present in the non-ketogenic ketogenic diet-booster composition as triglyceride form trihexanoin or glycerol trihexanoate. Trihexanoin is, if present, preferably present in the non-ketogenic ketogenic diet-booster composition in an amount to provide a daily dosage of trihexanoin in the range of 5 mg to 185 mg per kilogram bodyweight per day, preferably 35 mg to 90 mg per kilogram bodyweight per day, more preferably 50 mg to 75 mg per kilogram bodyweight per day.

In one embodiment the non-ketogenic ketogenic diet-booster composition comprises 2 mg to 20 g, more preferably 5 mg to 17 g C6 fatty acids or derivatives thereof per serving based on dry weight of the composition.

### Dietary Fibers as Source of Fatty Acids

In an alternative embodiment of the invention, the source of fatty acids are dietary fibers wherein C2, C4 and/or C6 fatty acids are provided by the microbial fermentation of these dietary fibers in the colon. In an embodiment the dietary fibers are provided in the non-ketogenic ketogenic diet-booster composition in an amount sufficient to provide C2, C4 and/or C6 fatty acids in the therapeutic effective range according to the invention to increase the ketone production of subjects on a ketogenic diet, sustain and/or improve ketosis and/or to prevent or treat tolerance to a ketogenic diet in subjects on a ketogenic diet.

In a preferred embodiment, dietary fibers in the non-ketogenic ketogenic diet-booster composition serve as a substrate for fatty acid production. The dietary fibers that serve as a substrate for fatty acids in the context of the invention are butyrogenic dietary fibers or butyrogenic dietary fiber blends. Butyrogenic dietary fibers or blends when fermented produce, more hexanoate and butyrate than propionate and pentanoate FA. For the sake of this specification, as the production of pentanoate and hexanoate are negligible anyway, the butyrogenic dietary fibers for use in the present invention are defined as dietary fibers or dietary fiber blends that produce butyrate and propionate in a beneficial ratio A beneficial weight ratio of butyrate to propionate as used herein is defined as more butyrate than propionate, i.e. a butyrate to propionate weight ratio of at least 1, preferably at least 1.1.

The butyrogenic dietary fibers that may serve as a substrate for generating fatty acids, preferably produce acetate, butyrate, propionate, and hexanoate, more preferably propionate and butyrate in a beneficial ratio, allowing to increase the ketone production of subjects on a ketogenic diet, sustain and/or improve ketosis and/or to prevent or treat tolerance to a ketogenic diet in subjects on a ketogenic diet.

The expression “a beneficial butyrate to propionate ratio” as used herein means that the weight of butyrate produced by fermentation of a fiber or mixture of fibers is at least equal or higher that the amount of propionate produced by the fermentation. For example, upon fermentation of locust bean gum or yeast beta-glucan more propionate than butyrate is produced, and these fibers thus have a butyrate to propionate ratio below 1 and cannot boost or increase ketone production. In contrast, soy fibers and inulin are exemplary fibers that upon fermentation have a beneficial butyrate to propionate ratio.

In a preferred embodiment the fermentation of butyrogenic dietary fibers beneficially results in a fatty acid production wherein butyrate and propionate are produced in a weight ratio of butyrate:propionate at least 1, preferably at least 1.1. Fermentation profiles of dietary fibers and concomitant butyrate to propionate ratio's from either a single source of fibers or blends of fibers, can be assessed using fermentation assays known in the art. Exemplary ways to assess the fermentation of fibers include using in vitro fermentation models with faecal sample pools of healthy adults with exposure to approximately 10 mg/ml fiber for 24 hours at 37° C. under anaerobic conditions.

In an embodiment there is provided between 1 g and 2 g of dietary fibers per 100 kcal of the total diet. In one embodiment in a diet of about 2500 kcal per day there may be provided between 25 and 50 g of butyrogenic dietary fibers that may provide for about 10 μM to about 250 μM of fatty acids including C4 and C6 fatty acids in the blood.

The dietary fibers that may provide C2, C4 and/or C6 fatty acids are fermentable dietary fibers, such as for example fermentable oligo- and polysaccharides. Preferably said dietary fibers are butyrogenic dietary fibers having a beneficial fermentation profile wherein butyrate and propionate are produced in a weight ratio of C4:C3 of at least 1, preferably at least 1.1. The fermentable fibers can include but are not restricted to pectins, mucilages, gums, galacto-oligosaccharides, oligofructan, inulin, polyfructoses, fructo-oligosaccharides, arabinoglactans, hemicelullose, resistant starch, soy fiber, oligosaccharides, or mixtures of thereof.

In one embodiment, the butyrogenic dietary fiber serving as a substrate for fatty acid production is a fermentable fiber which is selected in the group consisting of: pectins, mucilages, gums, galacto-oligosaccharides, oligofructan, inulin, polyfructoses, fructo-oligosaccharides, arabinogalactans, (hemi)celullose, resistant starch, soy fiber, oligosaccharides, and mixtures of thereof.

In a particular embodiment the use of fiber blends is preferred. Exemplary fiber blends for use in the context of the present invention are blends that are fermentable and provide a beneficial C4:C3 weight ratio of at least 1, preferably at least 1.1, even more preferably at least 1.5. An exemplary fiber blends is a fibre mixture that comprises a) beta-galactooligosaccharides, b) inulin, c) resistant starch and/or d) soluble soy polysaccharides provide fatty acids, in particular at a beneficial C4:C3 weight ratio.

A further exemplary fiber blend comprises a) FOS, b) inulin, c) soy fibre, d) resistant starch, e) acacia gum and/or f) cellulose. The blend is fermentable and provides fatty acids, in particular at a beneficial C4:C3 weight ratio of at least 1, preferably at least 1.1, even more preferably at least 1.5.

A further exemplary fiber blend comprises a) arabinoxylan, b) oat beta-glucan, c) pectin, and/or d) resistant starch. The fiber mixture is fermentable and produces butyrate and propionate in a weight ratio of C4:C3 of at least 1, preferably at least 1.1, even more preferably at least 1.5.

In an embodiment there is thus provided at least one of C2, C4 or C6 fatty acids or derivatives thereof, preferably at least C4 and additionally optionally C2 fatty acids for therapeutic use in
(i) enhancing ketosis and/or
(ii) preventing and/or treating resistance to a ketogenic diet, in a subject adhering to/taking a ketogenic diet, wherein the at least one of C4 or C6 fatty acids and optionally C2 fatty acids are comprised in a non-ketogenic ketogenic diet-booster composition, or the at least C4 or C6 fatty acids and optionally C2 fatty acids are provided in non-ketogenic amounts by a butyrogenic dietary fiber or butyrogenic dietary fiber blend comprised in a non-ketogenic ketogenic diet-booster composition wherein fermentation of said butyrogenic dietary fibers provides butyrate and propionate in a weight ratio of butyrate:propionate of at least 1, preferably at least 1.1.

Optionally the non-ketogenic ketogenic diet-booster can comprise one or more ingredients additional to the at least one of C4 or C6 fatty acids and optionally C2 fatty acids, for example an optional additional component selected from the group consisting of a protein, a carbohydrate, a vitamin, a mineral, an excipient, an emulsifier, a stabilizer, and mixtures thereof. The final non-ketogenic ketogenic diet-booster composition can be in a liquid format ready to be consumed, or in a powder format to be reconstituted in water before use. Preferably proteins form less than 30%, more preferably less than 20%, even more preferably less than 15% of the non-ketogenic ketogenic diet-booster composition based on weight of the composition. Preferably, if present, digestible carbohydrates form less than 20%, more preferably less than 15%, even more preferably less than 10% of the non-ketogenic ketogenic diet-booster composition based on weight of the composition.

In a further preferred embodiment, the non-ketogenic ketogenic diet-booster composition comprises C6 fatty acids. In a preferred embodiment both C4 and C6 fatty acids are comprised in the non-ketogenic ketogenic diet-booster composition

In an embodiment there is thus provided at least one of C4 or C6 fatty acids, preferably C4 and C6 fatty acids, and optionally C2 fatty acids for therapeutic use in
(i) enhancing ketosis and/or
(ii) preventing and/or treating resistance to a ketogenic diet, in a subject adhering to/taking a ketogenic diet, wherein the at least one of C4 or C6 fatty acids and optionally C2 fatty acids are comprised in a non-ketogenic ketogenic diet-booster composition, or the at least one of C4 or C6 fatty acids and optionally C2 fatty acids are provided in non-ketogenic amounts by butyrogenic dietary fiber or butyrogenic dietary fiber blend comprised in a non-ketogenic ketogenic diet-booster composition.

### Citric Acid

In a further embodiment the non-ketogenic ketogenic diet-booster composition may further comprise citric acid. Citric acid is converted to isocitrate in the first step in the Krebs cycle (also known as the citric acid or TCA cycle). Citric acid as used in the non-ketogenic ketogenic diet-booster composition is preferably in the form of a citrate salt with a counter-ion selected from to potassium (K⁺), sodium (Na⁺), Magnesium (Mg⁺) or calcium (Ca²⁺). In the context of the invention the amounts are calculated in terms of the corresponding (weight) amount of citrate.

In an embodiment there is thus provided at least one of C4 or C6 fatty acids and optionally C2 fatty acids for use in
(i) enhancing ketosis and/or
(ii) preventing and/or treating resistance to a ketogenic diet, in a subject adhering to/taking a ketogenic diet, wherein the at least one of C4 or C6 fatty acids and optionally C2 fatty acids are comprised in a non-ketogenic ketogenic diet-booster composition, or the at least one of C4 or C6 fatty acids and optionally C2 fatty acids are provided in non-ketogenic amounts by a dietary fibre blend comprised in a non-ketogenic ketogenic diet-booster composition, wherein the non-ketogenic ketogenic diet-booster composition optionally further comprises citrate.

In an embodiment, when citrate is present in the non-ketogenic ketogenic diet-booster composition, the citrate is present in therapeutically effective dose to increase the ketone production of subjects on a ketogenic diet, sustain and/or improve ketosis and/or to prevent or treat tolerance to a ketogenic diet in subjects on a ketogenic diet.

Citrate is, if present, preferably present in the non-ketogenic ketogenic diet-booster composition in an amount to provide a daily dosage of citrate in the range of 0.01 gram to 0.4 gram per kilogram bodyweight per day, preferably 0.04 gram to 0.15 gram per kilogram bodyweight per day, more preferably 0.08 gram to 0.1 gram per kilogram bodyweight per day.

Daily intake of citrate of less than 15 g/day for K+/Na+/Mg+-citrate and possibly less than 20 g/day for Ca²⁺-citrate is considered safe and does not give rise to a risk of electrolyte imbalances.

In one embodiment the non-ketogenic ketogenic diet-booster composition comprises 5 mg to 20 g, more preferably 10 mg to 15 g citrate per serving based on dry weight of the composition.

In an embodiment the non-ketogenic ketogenic diet-booster composition comprises C4 fatty acids, C6 fatty acids and citrate.

### Nicotinamide Riboside

The present disclosure provides non-ketogenic ketogenic diet-booster compositions further comprising a NAD+ precursor selected from the group consisting of tryptophan, nicotinic acid (niacin), nicotinamide (niacinamide, NAM), nicotinic acid riboside (NaR), nicotinamide riboside (NR), and mixtures thereof. In a preferred aspect the non-ketogenic ketogenic diet-booster composition may comprise nicotinic acid, nicotinamide riboside and/or nicotinamide, preferably nicotinamide riboside and/or nicotinamide, more preferably nicotinamide riboside (NR). In the context of the invention the amounts are calculated in terms of the corresponding (weight) amount of nicotinamide riboside. Equivalent amounts of nicotinamide calculated on the basis of the weight of NR are foreseen throughout the application.

As used herein, “nicotinamide riboside” includes derivatives thereof such as L-valine and L-phenylalanine esters of nicotinamide riboside.

In an embodiment there is thus provided at least one of C4 or C6 fatty acids and optionally C2 fatty acids for use in
(i) enhancing ketosis and/or
(ii) preventing and/or treating resistance to a ketogenic diet, in a subject adhering to/taking a ketogenic diet, wherein the at least one of C4 or C6 fatty acids and optionally C2 fatty acids are comprised in a non-ketogenic ketogenic diet-booster composition, or the at least one of C4 or C6 fatty acids and optionally C2 fatty acids are provided in non-ketogenic amounts by butyrogenic dietary fiber or a butyrogenic dietary fibre blend comprised in a non-ketogenic ketogenic diet-booster composition, wherein the non-ketogenic ketogenic diet-booster composition optionally further comprises a NAD+ precursor selected from nicotinic acid, nicotinamide riboside and/or nicotinamide, preferably nicotinamide riboside and/or nicotinamide.

In an embodiment, when nicotinamide riboside or nicotinamide is present in the non-ketogenic ketogenic diet-booster composition, the nicotinamide riboside or nicotinamide is present in therapeutically effective dose to increases the ketone production of subjects on a ketogenic diet, sustain and/or improve ketosis and/or to prevent or treat tolerance to a ketogenic diet in subjects on a ketogenic diet.

Nicotinamide riboside is, if present, preferably present in the non-ketogenic ketogenic diet-booster composition in an amount to provide a daily dosage of nicotinamide riboside in the range of 0.6 mg to 14.5 mg per kilogram bodyweight per day, preferably 1.4 mg to 4.5 per kilogram bodyweight per day, more preferably 2.5 to 3.5 mg per kilogram bodyweight per day.

In one embodiment the non-ketogenic ketogenic diet-booster composition comprises 0.2 mg to 2 g, more preferably 0.5 mg to 1.5 g nicotinamide riboside per serving based on dry weight of the composition.

In an embodiment the non-ketogenic ketogenic diet-booster composition comprises C4 fatty acids, C6 fatty acids, citrate and a NAD+ precursor.

### Non-Ketogenic Ketogenic Diet-Booster Composition Administration Forms

The non-ketogenic ketogenic diet-booster composition of the invention may be in any form suitable for enteral, oral or parenteral administration. Non-limiting examples of parenteral administration include intravenously, intramuscularly, intraperitoneally, subcutaneously, intraarticularly, intrasynovially, intraocularly, intrathecally, topically, and inhalation.

In one aspect of the present invention, the composition according to the invention may be used as a pharmaceutical product comprising one or more pharmaceutically acceptable carrier materials.

The composition according to the invention may in a preferred aspect be used as a nutritional product, for example as a nutritional supplement, e.g., as an additive to add to a ketogenic diet, as a fortifier, as a supplement to use in addition to a ketogenic diet.

The supplement, preferably for enteral application, may be a solid or liquid galenical formulation. Examples of solid galenical formulations are tablets, capsules (e.g., hard or soft shell gelatine capsules), pills, sachets, powders, granules and the like which contain the active ingredient together with conventional galenical carriers. Any conventional carrier material can be utilized. The carrier material can be organic or inorganic inert carrier material suitable for oral administration. Carrier materials may form up to 25 wt % of the supplement, preferably up to 20 wt %, more preferably up to 10 wt %. Additionally, additives such as flavouring agents, preservatives, stabilizers, emulsifying agents, buffers, and the like may be added in accordance with accepted practices of nutritional and pharmaceutical compounding.

The composition according to the invention may also be used as a nutritional product for use together with tube feeding, e.g. as an additive to a tube feed for intermittent or continuous administration.

The non-ketogenic ketogenic diet-booster composition may contain the daily dosage in one or more dosage units. The dosage unit may be in a liquid form or in a solid form, wherein in the latter case the daily dosage may be provided by one or more solid dosage units, e.g., in one or more capsules or tablets. The booster composition can be administered at the same time as the ketogenic diet or separated by a time interval or continuous.

In an embodiment, dosing of the non-ketogenic ketogenic diet-booster composition is at least monthly, preferably at least weekly, preferably at least daily; for example, a subject may receive one or more doses or servings daily, preferably 1 to 6 times per day, more preferably 3 to 4 times per day. In some embodiments, the administration continues for the remaining life of the individual. In specific embodiments, the administration occurs until a detectable improvement in ketosis occurs and, in further cases, continues to remain ameliorated.

The ideal duration of the administration of the composition can be determined by those of skill in the art.

In a particular non-limiting example, the daily doses for a 70 kg subject consuming 3000 kcal per day of a ketogenic diet having a ketogenic ratio of 1:1 to 4:1 can be as follows:
C2 fatty acid or derivative thereof (triacetin): (0.5 mg to 36 mg/kg/day)*70 kg=35 mg/day to 2520 mg/day;
C4 fatty acid or derivative thereof (tributyrin): (0.2 mg to 14 mg/kg/day)*70 kg=7 mg/day to 980 mg/day;
C6 fatty acid or derivative thereof (trihexanion): (6.5 mg to 185 mg/kg/day)*70 kg=455 mg/day to 12.95 g/day;
Citric acid (0.01 g to 0.4 g/kg/day)*70 kg=0.7 g/day to 28 g/day;
NR (0.6 mg to 14.5 mg/kg/day)*70 kg=42 mg/day to 1015 mg/day.

In a further embodiment there is provided the non-ketogenic, ketogenic diet-booster composition consisting essentially of
(i) 1 mg to 1.2 g C4 fatty acids/day; and optionally one or more of
(ii) 32.5 mg to 17 g C6 fatty acids/day;
(iii) 0.05 g to 20 g citrate/day; or
(iv) 3 mg to 1.45 g nicotinamide riboside/day. The ranges refer to the total amounts per 24 hours.

In a further embodiment there is provided the non-ketogenic, ketogenic diet-booster composition consisting essentially of
(i) 1 mg to 1.2 g C4 fatty acids/day; and optionally one or more of
(ii) 32.5 mg to 17 g C6 fatty acids/day;
(iii) 0.05 g to 20 g citrate/day; or
(iv) 3 mg to 1.45 g nicotinamide riboside or equivalent amounts of nicotinamide/day; or
(v) 2.5 mg to 3 g C2 fatty acids/day. The ranges refer to the total amounts per 24 hours.

In a further embodiment there is provided the non-ketogenic, ketogenic diet-booster composition consisting essentially of
(i) 1 mg to 1.2 g C4 fatty acids/day; and,
(ii) 32.5 mg to 17 g C6 fatty acids/day;
and optionally one or more of
(iii) 0.05 g to 20 g citrate/day; or
(iv) 3 mg to 1.45 g nicotinamide riboside or equivalent amounts of nicotinamide/day. The ranges refer to the total amounts per 24 hours.

In an embodiment there is provided the non-ketogenic, ketogenic diet-booster composition consisting essentially of
(i) 0.2 mg to 1.2 g C4 fatty acids;
(ii) 5 mg to 17 g C6 fatty acids;
(iii) 10 mg to 20 g citrate; and
(iv) 0.5 mg to 1.5 g nicotinamide riboside per serving based on dry weight of the composition. Equivalent amounts of nicotinamide calculated on the basis of the weight of NR are foreseen as nicotinamide riboside.

In a further embodiment there is provided the non-ketogenic, ketogenic diet-booster composition consisting essentially of
(i) 0.2 mg to 1.2 g C4 fatty acids;
(ii) 5 mg to 17 g C6 fatty acids;
(iii) 10 mg to 20 g citrate;
(iv) 0.5 mg to 1.5 g nicotinamide riboside per serving, further optionally comprising
(v) 0 to 3 g C2 fatty acids, preferably 0.5 mg to 3 g C2 fatty acids.

In an embodiment there is provided the non-ketogenic, ketogenic diet-booster composition, intended for administration 4 to 6 times per day, consisting essentially of
(i) 0.1-0.3 mg C4 fatty acids;
(ii) 4.6-5.4 mg C6 fatty acids;
(iii) 9-11 mg citrate; and
(iv) 0.4-06 mg nicotinamide riboside per serving.

In a further embodiment there is provided the non-ketogenic, ketogenic diet-booster composition, intended for administration once daily, consisting essentially of
(i) about 1.2 g C4 fatty acids;
(ii) about 17 g C6 fatty acids;
(iii) about 20 g citrate;
(iv) about 1.5 g nicotinamide riboside per serving. Optionally, the non-ketogenic, ketogenic diet-booster composition, intended for administration once daily further includes (v) about 3 g C2 fatty acids.

Said non ketogenic, ketogenic diet-booster composition is formulated in the form of a dietary supplement to a ketogenic diet. Typically a serving of the non-ketogenic, ketogenic diet-booster composition is between about 10 g and 25 g.

The invention further pertains to a non-ketogenic ketogenic diet-booster composition essentially consisting of
(i) 4 mg to 480 mg C4 fatty acids;
(ii) 260 mg to 3.3 g C6 fatty acids;
(iii) 0.2 mg to 4.4 g citrate; and
(iv) 12 mg to 286 mg nicotinamide riboside,
(v) 20 mg to 1.2 g C2 fatty acids;
wherein the weight ratio of C2 and C4 fatty acids:C6 fatty acids is between 1:1.8 and 1:2.1, preferably between 1:1.9 and 1:2.0;
the weight ratio of C2 and C4 fatty acids:citrate is between 1:2.4 and 1:2.7, preferably between 1:2.5 and 1:2.6 and
the weight ratio of C2 and C4 fatty acids:nicotinamide riboside is between 1:0.15 and 1:0.19, preferably between 1:0.16 and 1:0.18 based on dry weight and wherein the total weight of the composition is between about 10-20 grams.

Further there is provided for a kit of parts comprising 1) the non-ketogenic, ketogenic diet-booster composition and 2) one or more nutritional products having a ketogenic ratio between 1:1 and 4:1. In a further aspect, the present disclosure provides kits suitable for administering food compositions to a subject in need of ketosis. There is also provided for said kit of parts for use in (i) enhancing ketosis associated with a ketogenic diet and/or (ii) preventing and/or treating resistance to a ketogenic diet, in a subject adhering to a ketogenic diet.

### EXAMPLES

For a more complete understanding of the present disclosure, reference is now made to the following examples taken in conjunction with the accompanying drawings.

### Example 1

An assay to measure ketone production in mouse liver cells (Hepa1-6 cell line) was used wherein cells were first grown to a monolayer (growth phase), then exposed to starvation to deplete cellular lipid stores and subsequently brought into a ketogenic phase wherein cells were cultured in presence of oleic acid or a ketogenic diet fat blend with or without the presence of one of C2 fatty acid, C4 fatty acid, C6 fatty acid, citric acid and nicotinamide riboside. The ketone body production was subsequently determined by measurement of beta-hydroxybutyrate in the cell culture medium according to the details set out below:

Growth phase: Cells were seeded in 6-well plates at a concentration of 90700 cells/cm² and allowed to grow for 24 hours at 37° C. in Dulbecco's Modified Eagle Medium (DMEM) supplemented with 5 mM glucose, 10% Fetal Bovine serum (FBS), 4 mM L-glutamine, 1 mM pyruvate, and antibiotics (1% penicillin & streptomycin).

Starvation phase: At the end of the growth phase the cells formed a monolayer. The medium was exchanged for DMEM containing 1 mM glucose and antibiotics (1% penicillin & streptomycin) but no FBS. The medium was further supplemented with L-carnitine (a required co-factor for the uptake of fatty acids into the mitochondria, where fatty acids are turned into ketones). The cells were incubated in this medium for 24 hours. During this time, the cells were vastly deprived of glucose, lipids and hormones contained in FBS, forcing them to consume and empty their lipid stores. Thereby, any interference with the nutrient testing was avoided. Moreover, the starvation phase is thought to induce specific metabolic changes that maximize the production of ketones.

Ketogenic phase: At the end of the starvation phase, the medium was exchanged for Krebs-Henseleit buffer (KHB) supplemented with the L-carnitine and the test nutrients (table 1, single fatty acids C2, C4, C6 and citrate, nicotinamide riboside in combination with oleic acid fatty acid blend).

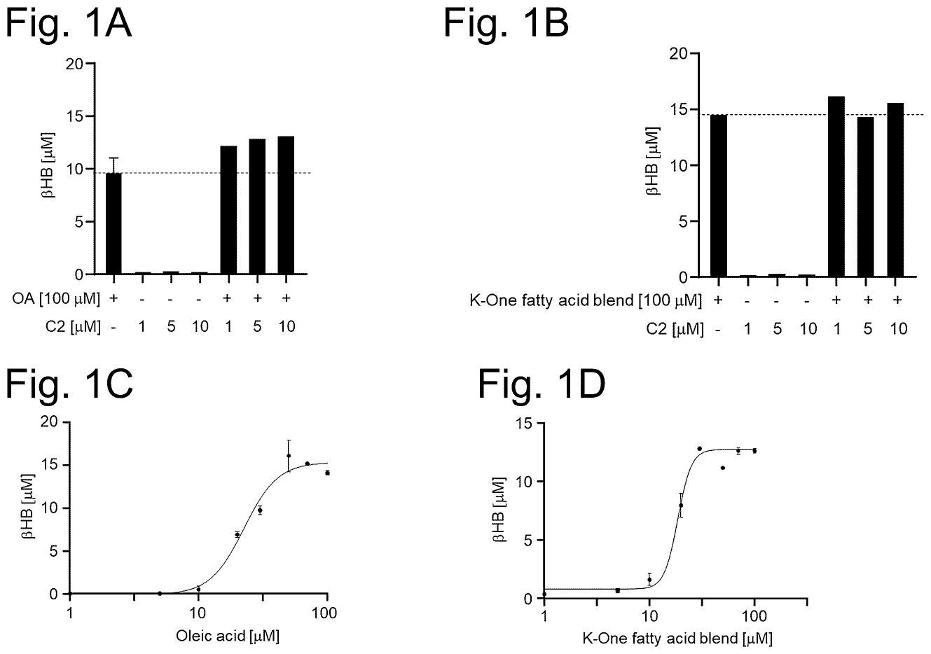

The fatty acid blend has a similar fatty acid composition as the one used in a ketogenic diet (K-one). The content of every fatty acid in the blend is given as the molar ratio to Oleic acid (OA), whose molar content is set to 1. Concentrations of individual fatty acids in 100 μM of the blend are shown in Table 1. Dilutions of this blend were used to determine the dose response curve in FIGS. 1B and 1D. C2 and C4 are not included in the blend.

TABLE 1
Composition of fatty acid blend.

| | | | Concentration (μM) |
|---|---|---|---|
| | Lipid | molar | in 100 μM total |
| Ingredient | numbers | ratio | fatty acid blend. |
| caproic acid | C-6:0 | 0.0015 | 0.05 |
| caprylic acid | C-8:0 | 0.0463 | 1.72 |
| capric acid | C-10:0 | 0.0363 | 1.35 |
| lauric acid | C-12:0 | 0.1071 | 3.98 |
| myristic acid | C-14:0 | 0.0682 | 2.53 |
| palmitic acid | C-16:0 | 0.9354 | 34.73 |
| stearic acid | C18:0 | 0.1184 | 4.39 |
| oleic acid | C18:1ω9 | 1.0000 | 37.13 |
| linoleic acid | C18:2ω6 | 0.2618 | 9.72 |
| alpha-linolenic acid | C18:3ω3 | 0.0274 | 1.02 |
| eicosapentaenoic acid | C20:5ω3 | 0.0168 | 0.62 |
| docosahexaenoic acid | C22:6ω3 | 0.0741 | 2.75 |

KHB is composed of salts and bicarbonate as a chemical buffer to maintain the osmotic pressure and acidity (pH) at physiological levels. Medium and long chain fatty acids are coupled to bovine serum albumin prior to the assay to make them soluble in KHB and allow their efficient uptake by the cells. The coupling is physiological since in humans, medium and long chain fatty acids are normally coupled to albumin for transport in the blood circulation. Short chain fatty acids (C2, C4) were not coupled to albumin in the assay as they are water-soluble and circulate freely in humans. The cells were incubated for 6 hours at 37° C. During this time they produce ketones from the test nutrients with little to no interference from other nutrients and secrete β-hydroxybutyrate (β) into the medium.

β-Hb measurements: At the end of the ketogenic phase, the whole medium was collected and dried by vacuum centrifugation for 20 hours at 21° C. The dry pellet was resuspended in a small volume of assay buffer to yield a 10-20-fold higher β-Hb concentration than in the medium. The samples were deproteinized using spin columns to measure the free unbound fraction of β-Hb. The β-Hb concentration was then measured by an enzymatic reaction that generates a fluorescent product (Cayman Chemicals 700740). The Fluorescence was measured on a fluorescence microplate reader and is directly proportional to the β-Hb concentration in the sample within a range of 0.1 μM to 50 μM β-Hb.

### 1.1 Effect of C2 on Ketone Production

C2 provided alone to liver cells at a concentration of 1 μM, 5 μM and 10 μM yielded virtually no bHB (FIGS. 1A and 1B). When liver cells were incubated with a concentration range of oleic acid (OA), a long chain fatty acid, the dose-response curve showed that the top-level production of ketones from incubation with oleic acid (OA) alone was achieved at a concentration of 100 μM OA (FIG. 1C). The fatty acid plasma concentrations during a ketogenic diet treatment are usually above 100 μM in humans. Therefore, to assess the ketogenic enhancement by C2, OA was assayed at 100 μM. Combining 1 μM, 5 μM, or 10 μM C2 with 100 μM OA yielded more βHb than 100 μM OA alone. The effect of combining C2 and OA on βHb production was more than additive, it cannot be explained by the sum of the individual effects of C2 and OA on βHb production. Therefore, C2 at a physiological concentration of 1 μM to 10 μM potentiates the top-level production of ketones from a single fatty acid.

The same experiment was performed with a fatty acid blend composed of medium, long and very long chain fatty acids and contained in a ketogenic diet (denoted as K-one). The composition of the blend is shown in table 1 and does not contain C2. When liver cells were incubated with a concentration range of this blend, the dose-response curve showed that production of βHb from this blend was maximized at a total fatty acid concentration of 100 μM (FIG. 1D).

The fatty acid concentrations of humans on a ketogenic diet in plasma exceeds 100 μM, meaning that the top-level production of ketones is then achieved. Therefore, to assess the ketogenic enhancement by C2, the fatty acid blend was tested at a concentration of 100 μM. Combining 1 μM or 10 μM C2 with 100 μM fatty acid blend led to slightly greater production of βHb than an equimolar concentration of the fatty acid blend alone (FIG. 1B). The effect of combining C2 and the fatty acid blend on βHb production was more than additive since C2 alone yielded no βHb (FIG. 1B). Therefore, in a physiological concentration range of 1 μM to 10 μM, C2 potentiates the top-level production of βHb from a fatty acid blend contained in a ketogenic diet.

In summary, C2 within a low physiological concentration of 1 μM to 10 μM enhances the production of ketones (βHb) from a single fatty acid (oleic acid, OA) (FIG. 1A) and from a blend of fatty acids contained in a ketogenic diet (K-one) (FIG. 1B). The effect of C2 is not explained by it being used as a substrate since it does not yield any ketones when provided alone to liver cells. Furthermore, the single fatty acid (oleic acid) and the fat blend (K-one) were tested with C2 at a concentration of 100 μM, when they both maximize ketone production on their own (FIGS. 1C, D). Therefore, C2 itself does not serve as a substrate for ketone production but boosts the top-level production of ketones from a single fatty acid or a blend of fatty acids to a level higher than obtained without the presence of C2.

### 1.2 Effect of C4 on Ketone Production

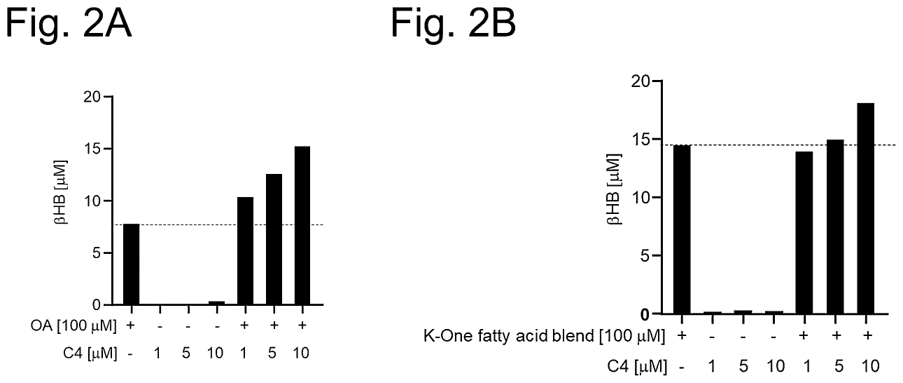

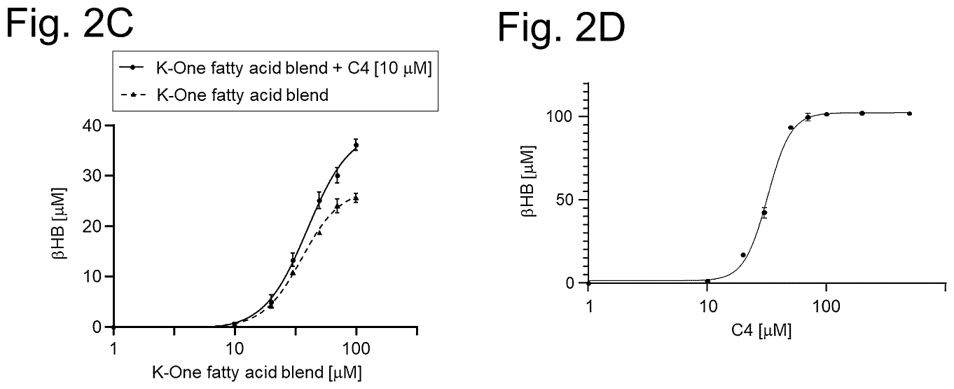

Exposing liver cells to C4 alone at a concentration of 1 μM, 5 μM and 10 μM hardly yielded any βHb (FIGS. 2A and 2B). The top-level production of ketones from incubation with OA alone in the assay was achieved at a concentration of 100 μM OA (FIG. 1C). Since fatty acid plasma concentrations are usually above 100 μM in humans on a ketogenic diet, OA was assayed at a concentration of 100 μM. Combining 1 μM, 5 μM, or 10 μM C4 with 100 μM OA yielded more βHb than providing 100 μM OA alone (FIG. 2A). Moreover, the βHb production increased with the concentration of C4 provided. The effect of combining C4 and OA on βHb production was more than additive, it cannot be explained by the sum of the individual effects of C4 and OA on βHb production. Therefore, C4 given in a physiological concentration range of 1 μM to 10 μM potentiates the top-level production of ketones from a single fatty acid.

Next, the ketogenic enhancement by C4 of the ketone production from a fatty acid blend contained in a ketogenic diet (K-one) was assessed. This fatty acid blend did not contain C4. Combining 10 μM C4 with 100 μM fatty acid blend produced more βHb than providing the 100 μM fatty acid blend alone (FIG. 2B). The increase in βHb production achieved by combining 10 μM C4 and 100 μM fatty acid blend was more than additive since providing C4 alone yielded no βHb (FIG. 2B).

Liver cells were also co-incubated with a fixed concentration of 10 μM C4 and a concentration range of 1 μM to 100 μM of a fatty acid blend contained in a ketogenic diet (K-one). Control liver cells were exposed to the same concentration range of this fatty acid blend in the absence of C4. C4 increasingly enhanced the production of ketones from the fatty acid blend as the fatty acid blend concentration neared 100 μM (FIG. 2C). These results demonstrate that a low physiological concentration of C4 potentiates the production of βHb from a fatty acid blend contained in a ketogenic diet.

To assess whether C4 may be used as a substrate for ketone synthesis at concentrations of 10 μM and below, a dose-response curve for C4 as a substrate for the production of βHb was obtained, covering a concentration range for C4 from 1 μM to 100 μM. The dose-response curve shows that C4 starts to be turned into βHb above a concentration of 10 μM (FIG. 2D). This further corroborates that C4 at concentrations below 10 μM acts as a functional enhancer of the production of ketones from a single fatty acid or a fat blend and not as a direct substrate for ketone synthesis.

Overall, it was found that C4 within a low physiological concentration range of 1 μM to 10 μM potentiates the production of ketones (βHb) from a single fatty acid (oleic acid, OA) (FIG. 2A) and from a blend of fatty acids contained in a ketogenic diet (K-one) (FIG. 2B). The effect of C4 on total ketone production is clearly greater than the sum of the individual contributions of equimolar concentrations of C4 and the fat blend to the total ketone yield. Hence, it is more than additive and therefore not explained by C4 being used as a substrate for ketone production within a concentration range of 1 μM to 10 μM. This is also supported by the dose-response curve for C4, which shows that C4 starts to be effectively turned into ketones at concentrations above 10 μM (FIG. 2D). Therefore, C4 within a low physiological concentration range of 1 μM to 10 μM potentiates the top-level production of ketones from a single fatty acid or a blend of fatty acids.

### 1.3 Effect of C6 on Ketone Production

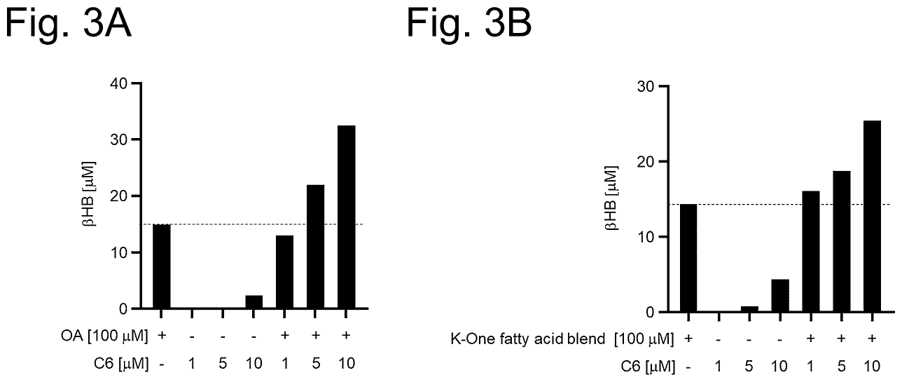

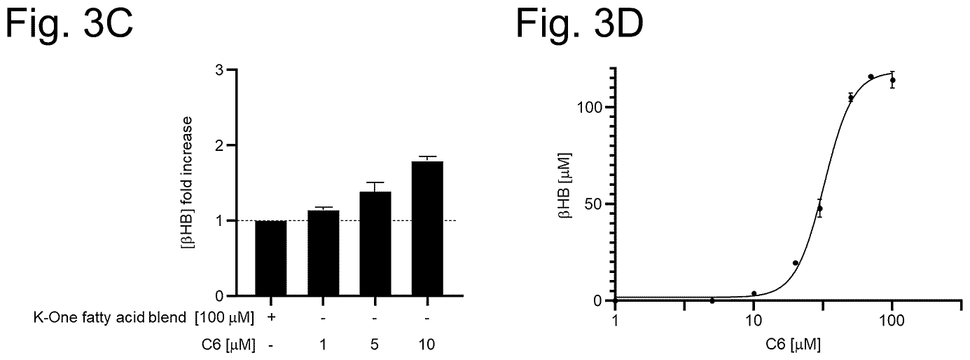

It was found that when performing similar experiments as described in more detail above for C2 and C4 that C6 within a low physiological concentration range of 1 μM to 10 μM potentiates the production of ketones (βHb) from a single fatty acid (oleic acid, OA) (FIG. 3A) and from a blend of fatty acids contained in a ketogenic diet (K-one) (FIGS. 3B and 3C). The effect of C6 on total ketone production from the fat blend was greater than the sum of the individual contributions of equimolar concentrations of C6 and the fat blend to the total ketone yield. Hence, it is more than additive and therefore not explained by C6 being used as a substrate for ketone production within a concentration range of 1 μM to 10 μM. This is also supported by the dose-response curve for C6, which shows that C6 starts to be effectively turned into ketones at concentrations above 10 μM (FIG. 3 D). The data thus indicate that C6 functionally upregulates ketone production within a concentration range of 1 μM to 10 μM. Both, the single fatty acid (oleic acid) and the K-one fat blend were tested at a concentration of 100 μM, when they both maximize ketone production on their own (FIGS. 1C and 1D). The K-one fat blend contains a trace amount of C6 (Table 1). However, the concentration of C6 in a 100 μM K-one blend is 20- to 200-fold below the 1 μM to 10 μM concentration range of C6 at which it enhanced ketogenesis from the blend. Although it is possible that this trace amount of C6 in the K-one fat blend could have already stimulated ketogenesis, our data show that adding C6 within a concentration range of 1 μM to 10 μM further potentiates the top-level production of ketones from a fatty acid blend. In summary, C6 at low physiological concentrations between 1 μM and 10 μM functionally enhances the production of ketones from a single fatty acid or a blend of fatty acids.

### 1.4 Effect of Citrate on Ketone Production

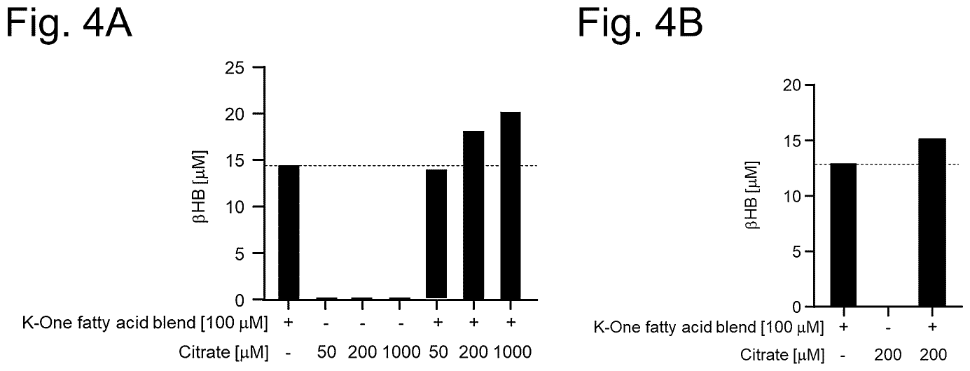

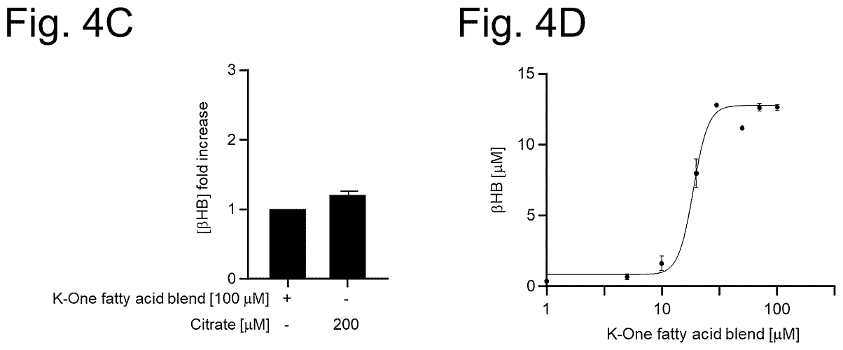

It was found that when performing similar experiments as described in more detail above for C2 and C4 that citrate within a physiological to supraphysiological concentration range of 200 μM to 1000 μM increases the production of ketones (βHb) from a blend of fatty acids contained in a ketogenic diet (K-one) (FIG. 4A-C). The K-one fat blend does not contain citrate (Table 1). The fat blend was tested at a concentration of 100 μM, when it achieves top level ketone production on its own (FIG. 1D). The effect of citrate on total ketone production from this fat blend was clearly greater than the sum of the ketone productions achieved individually by the same (equimolar) amounts of citrate or the fat blend (FIGS. 4A and 4B). In fact, providing citrate alone yielded no ketones but when added to the fat blend, it increased the ketone production from the fat blend by ˜10-20% (FIG. 4A-C; FIG. 4C shows the average for 200 μM citrate of experiment shown in FIGS. 4A and 4B, *P<0.05). Hence, citrate potentiates the top-level production of ketones from a blend of fatty acids contained in a ketogenic diet.

### 1.5 Effect of Nicotinamide Riboside on Ketone Production

A ketogenic diet treatment leads to great consumption of NAD+ and other metabolically relevant NAD+ derivatives such as NADP+ creating a state of NAD+ exhaustion. Therefore, it was hypothesized that nicotinamide riboside (NR) may replenish NAD+ and thus sustains ketone production more effectively in liver cells than other NAD+ precursors including nicotinamide (NAM), nicotinic acid (NA), Niacin (defined as the combination of NA and NAM) or the amino acid tryptophan (Trp). While NA and NAM have been commonly referred to as vitamin B3, NR is a recently discovered and naturally occurring vitamin B3 member. An assay was developed to measure NAD+ replenishment of NAD+ depleted mouse liver cells (Hepa1-6 cells) by NAD+ precursors under ketogenic diet-like conditions. In this assay, mouse liver cells were starved of glucose and depleted of NAD+ through incubation in a very low glucose (1 mM) medium free of vitamin B3, tryptophan (Trp) and FBS and supplemented with 10 nM of an NAD+ depleting drug called FK866. Thereafter, the cells were fed with NR or NAM or Niacin (combination of NAM and NA in a 1:1 molar ratio) or Trp to replenish NAD+. At the end of the supplementation phase, the cells were scraped and extracted using the NAD+ extraction buffer of the EnzyChrom™ NAD/NADH Assay Kit from BioAssay Systems. The NAD+ content of the cells was determined with the same kit following the manufacturer's manual. The NAD+ measurement is based on a lactate dehydrogenase reaction, in which NAD+ is reduced to NADH, which in turn reduces a formazan (MTT) reagent. The intensity of the reduced product colour, measured at 565 nm, is proportional to the NAD+ concentration in the sample. This assay is highly specific for NAD+ and has minimal interference (<1%) by NADP+ or NADPH.

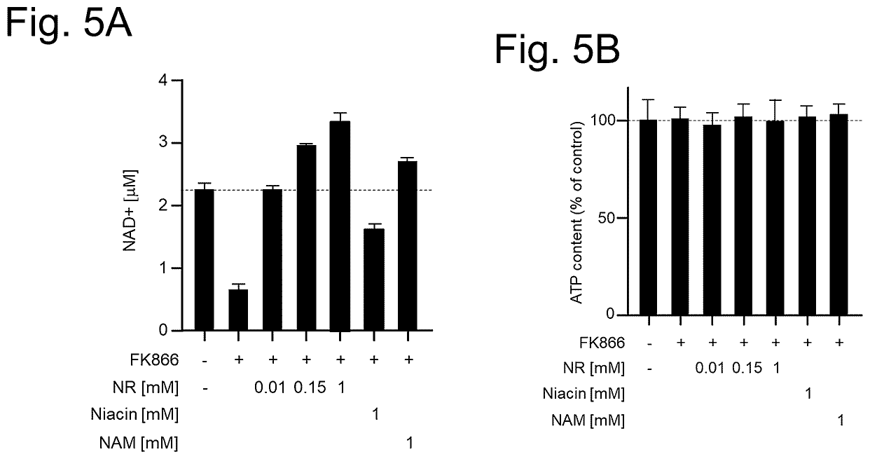

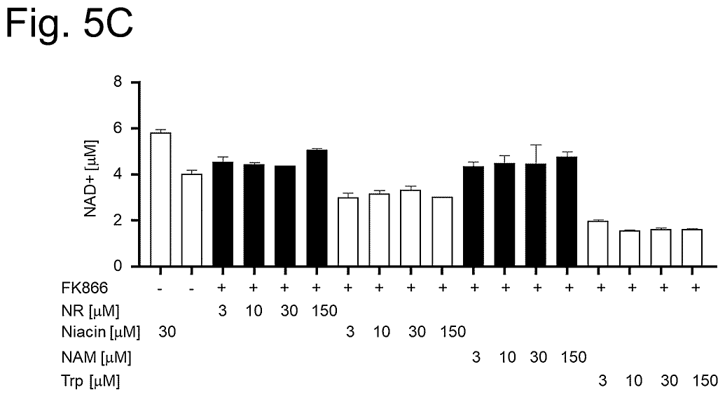

It was found that FK866 treatment significantly depletes liver cells of NAD+ by ˜70% of the baseline NAD+ content of untreated liver cells (FIG. 5A, P<0.0001). Furthermore, it was found that NR provision dose-dependently replenishes NAD+ content to and above this baseline within 24 hours of treatment (FIG. 5A). Additionally, it was found that NR increases the NAD+ content of NAD+ depleted liver cells to significantly greater extent than equimolar amounts of NAM or Niacin at high concentrations of 1 mM (FIG. 5A) and to greater extent than equimolar amounts of Niacin at lower concentrations below 150 μM (FIG. 5C). These differences are not due to changes in cell viability as cell counts (determined by ATP content measurements) were equal between different treatments (FIG. 5B). Hence, NR more effectively replenishes NAD+ than NAM and Niacin in NAD+ depleted mouse liver cells.

To measure the impact of replenishing NAD+ in liver cells with NR on ketone production, mouse liver cells were nutrient-deprived in medium containing low (1 mM) glucose, no fetal bovine serum, and no NAD+ precursors in the form of vitamin B3 (Niacin, NA, NAM) or Trp, but supplemented with L-carnitine.

Concomitantly the cells were treated with FK866. These conditions created ketogenic diet like nutrient conditions and depleted the cells of NAD+ After 18 hours of incubation, FK866 was removed from the medium and a concentration range of NR was added for the replenishment of NAD+. After 24 hours, the cells were switched to Krebs-Henseleit buffer (KHB) containing L-carnitine and a concentration range (1 μM to 100 μM) of oleic acid, a long chain fatty acid, for 6 hours to induce and measure the production and secretion of b-hydroxybutyrate (βHB) into the medium.

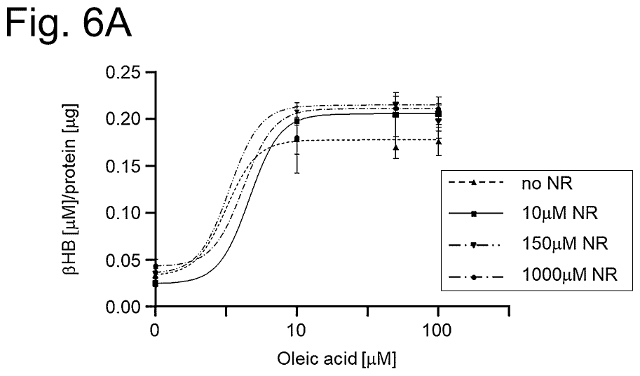

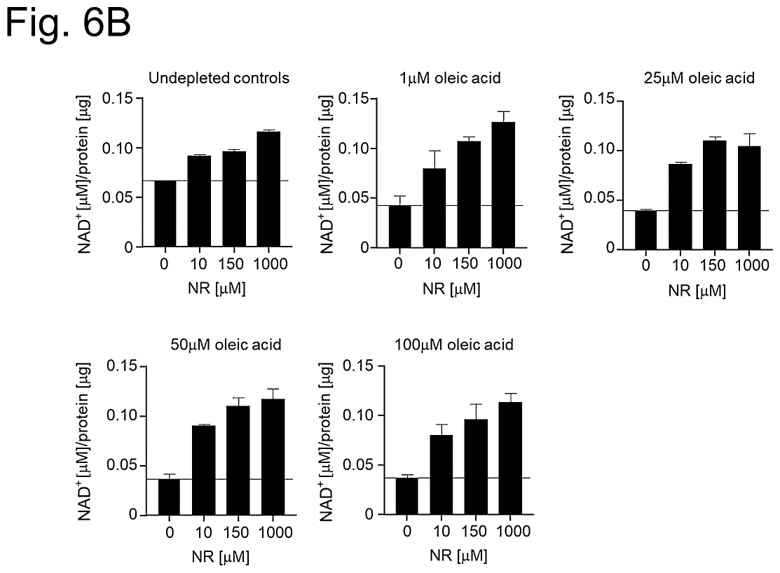

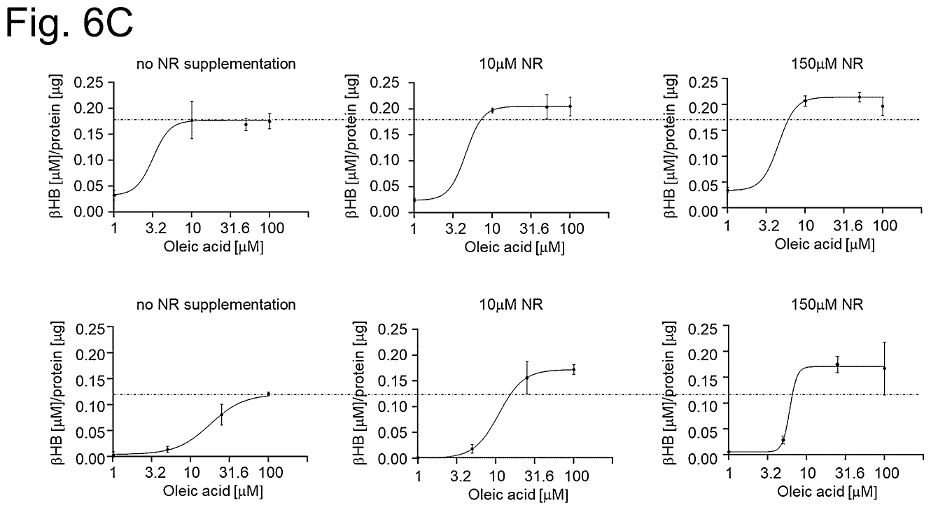

It was found that supplementing NAD+ depleted liver cells with NR at a concentration of 10 μM, 150 μM and 1000 μM increased the top-level production of ketones (FIGS. 6A and 6C) and their NAD+ content (FIG. 6B) under ketogenic diet like nutrient conditions. Combined with a greater ability to replenish NAD+ content (FIG. 5), it is concluded that NR more effectively sustains ketone production than NAM or Niacin or Trp.

### 1.6 Effect of the Combination of C4 and C6 on Ketone Production

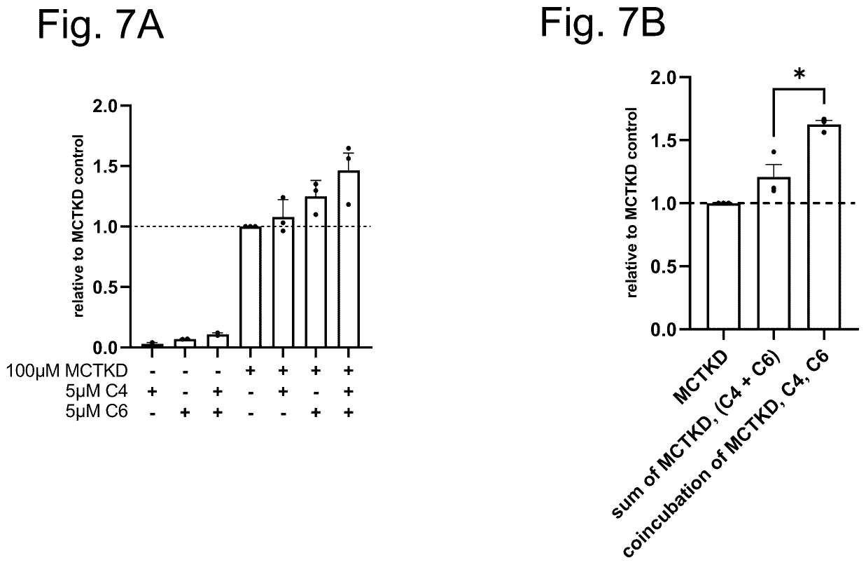

It was found that when performing similar experiments as described above for the single ingredients with a combination of C4 and C6 at a low physiologic amount of 5 μM each the production of ketones (βHb) from a blend of fatty acids contained in a ketogenic diet (K-one) (FIG. 7A) was enhanced in a more than additive manner. In the absence of a ketogenic fat blend providing C4 or C6 alone or in combination with each other yielded hardly any ketones, but when C4 and C6 were added in combination to the fat blend, it increased the ketone production from the fat blend by ˜40%. The observed increase was to an extent higher than could be predicted on the basis of the effect on ketone production observed for C4 and C6 individually (bars 3 and 4 versus the 6^{th} bar of FIG. 7A respectively). The dotted line represents the threshold ketone production induced by the ketogenic fat blend. FIG. 7B shows the synergy between the ketogenic fat blend and the combination of C4 and C6; the middle bar shows the expected effect on ketone production by adding up the sum of the effect on ketone production observed with C4 and C6 individually, the 3rd bar on the right-hand side shows the actual synergistic effect observed of coincubation of C4 and C6. In conclusion C4 and C6 together synergistically boost top-level ketone production from a ketogenic fat blend.

### 1.7. Effect of the Combination of C4, C6, Citrate and NR or NAM on Ketone Production

In a next assay the boosting or potentiating effect of 5 μM C4, 5 μM C6, 200 μM citrate either with 10 μM NR or 10 μM NAM was assessed.

The experimental set-up was similar to the set-up for the single ingredients as described here above with the exception that the medium during the starvation phase was adapted:

At the end of the growth phase when the cells formed a monolayer medium was exchanged for DMEM containing 1 mM glucose and antibiotics (1% penicillin & streptomycin) but no FBS. The medium was further supplemented with L-carnitine (a required co-factor for the uptake of fatty acids into the mitochondria, where fatty acids are turned into ketones). For the specific assessment of the effect of NR or NAM in the combination of ingredients, the medium was additionally deprived of Vitamin B3 and tryptophan since tryptophan can compensate for the lack of B3 as an NAD+ precursor.

At the end of the starvation phase, the medium was exchanged for Krebs-Henseleit buffer (KHB) supplemented with the L-carnitine and the test nutrients used were as follows:
i) full combination of C4, C6, citrate and NR
ii) full combination of C4, C6, citrate and NAM
iii) ketogenic fat blend
iv) full combination of C4, C6, citrate and NR with a ketogenic fat blend
v) full combination of C4, C6, citrate and NAM with a ketogenic fat blend

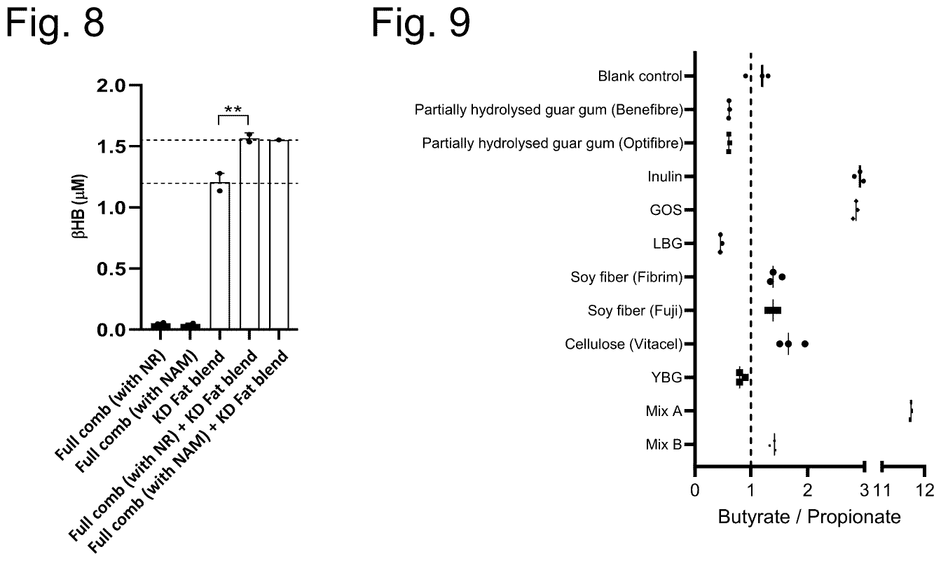

βHB production was subsequently assessed as described above. It was found that in the absence of a ketogenic fat blend both the combination with NR as the combination with NAM did not result in a substantial ketone production (FIG. 8). The use of a ketogenic fat blend alone resulted in the production of ketone production, reflected by the dashed line in the graph and herewith denominated the threshold level of ketone production. The further addition of either the combination with NR or the combination with NAM to the ketogenic fat blend resulted in a significant increase in ketone production above this threshold level. Surprisingly, the increase above the threshold level conveyed by either combination was greater than the ketone yields that is achieved individually by either combination in the absence of the ketogenic fat blend. Therefore, this result demonstrates a more than additive that is a synergistic effect of the combination with NR as with the combination with NAM on ketone production from a ketogenic fat blend. Therefore, the combination of C4, C6, citrate and either NR or NAM was found to potentiate top-level ketone production from a ketogenic fat blend.

### 2. Dietary Fiber Fermentation Assessment

A standard adult human gut microbiota pool was used to assess the fermentation of dietary fibers. This pool was established via fecal donations from 6 healthy adult volunteers (Caucasian individuals, age 25-60 years, no antibiotic use in the 3 months preceding the donation, self-assessment of health status). Before starting the fermentation with the test compounds, the standardized fecal adult pool was incubated in SIEM (standard ileal effluent medium) under anaerobic conditions overnight (37° C.; 300 rpm) in order to activate the bacteria as described in Schuren F et al. The i-screen: A Versatile Preclinical Platform for Gut Microbiota Studies. J Prob Health. 7:212, 2019.

The dietary fibers were tested at a concentration of 6-10 mg/ml, depending on their viscosity and/or fermentability. All fibers were tested in triplicates. The following conditions were applied:
Partially hydrolysed guar gum (Benefibre & Optifibre) 6.6 mg/ml
Inulin (Orafti HP) 10 mg/ml
GOS (Vivinal GOS) 6 mg/ml
Locust bean gum (LBG) 6 mg/ml
Soy fiber (Fibrim) 10 mg/ml
Soy fiber (Fuji) 10 mg/ml
Cellulose (Vitacel) 10 mg/ml
Yeast beta-glucan (YBG) 10 mg/ml
Mix A, containing GOS, inulin, soy fiber (Fuji), resistant starch (Novelose 330) 10 mg/ml
Mix B, containing FOS, inulin, soy fiber (Fibrim), resistant starch (Novelose 330), acacia gum, cellulose (Vitacel) 6.6 mg/ml

In each run a negative control was included (a blank control medium with only the faecal pool) and all conditions started at pH 6.3. After 24 hours of anaerobic fermentation in SIEM medium at 37° C., samples were collected for pH measurements and metabolite analysis.

The pH was measured by immersing a 423 pH-electrode (Mettler Toledo, Columbus, OH, USA), connected to a Handy-lab pH meter (Schott Glas, Mainz, Germany), directly in a sample.

SCFA covering acetate, propionate, and n-butyrate and branched chain fatty acids (BCFA) covering iso-butyrate and iso-valerate were analyzed as described by Jouany et al (1982) with modifications as described by Van Nuenen et al. (2003). Briefly, fermented material from the i-screen samples was centrifuged (˜12,000 g, 5 min). Cells were removed from the supernatant by filter sterilization (0.45 μm). A mixture of formic acid (20%), methanol and 2-ethyl butyric acid (internal standard, 2 mg/ml in methanol) was added. A 3 μl sample with a split ratio of 75.0 was injected on a GC-column (ZB5HT inferno, ID 0.52 mm, film thickness 0.10 μm; Zebron; Phenomenex, USA) in a Shimadzu GC-2014 gas chromatograph.

It was found that after 24 hr of fermentation inulin, GOS, the soy fibers, cellulose, and both mixes result in a higher butyrate/propionate ratio as compared to the blank control (above 1 and >1.1) (FIG. 9). Dietary fibers and mixtures of such fibers having a butyrate to propionate ratio of at least 1 are considered butyrogenic dietary fibers according to the invention. In contrast the partially hydrolysed guar gums, LBG, and YBG remain below a butyrate/propionate ratio of 1.

1. At least one of C4 or C6 fatty acids and optionally C2 fatty acids for therapeutic use in
(i) enhancing ketosis associated with a ketogenic diet and/or
(ii) preventing and/or treating resistance to a ketogenic diet,
In a subject adhering to a ketogenic diet, wherein the at least one of C4 or C6 fatty acids and optionally C2 fatty acids are comprised in a non-ketogenic ketogenic diet-booster composition, or the at least C4 or C6 fatty acids and optionally C2 fatty acids are provided in non-ketogenic amounts by a butyrogenic dietary fiber or butyrogenic dietary fiber blend comprised in a non-ketogenic ketogenic diet-booster composition.

2. The at least one of C4 or C6 fatty acids and optionally C2 fatty acids for use according to claim 1, wherein enhancing ketosis means increasing blood ketone concentrations in a subject on a ketogenic diet using the non-ketogenic ketogenic diet-booster composition in comparison to the blood ketone concentration in a subject consuming the same ketogenic diet and not using the non-ketogenic ketogenic diet-booster composition.

3. The at least one of C4 or C6 fatty acids and optionally C2 fatty acids for use according to claim 1, wherein the non-ketogenic ketogenic diet-booster composition further comprises one or more compounds selected from a) citric acid and/or b) nicotinamide riboside and/or nicotinamide.

4. The at least one of C4 or C6 fatty acids and optionally C2 fatty acids for use according to claim 1, wherein the non-ketogenic ketogenic diet-booster composition is in the form of a nutritional supplement.

5. The at least one of C4 or C6 fatty acids and optionally C2 fatty acids for use according claim 1,
wherein the at least C4 or C6 fatty acids and optionally C2 fatty acids are provided in non-ketogenic amounts by a butyrogenic dietary fibre or butyrogenic dietary fiber blend, and
wherein the dietary fibers are selected from pectins, mucilages, gums, galacto-oligosaccharides, oligofructan, inulin, polyfructoses, fructo-oligosaccharides, arabinoglactans, hemicelullose, resistant starch, fuji soy, oligosaccharides, or mixtures of thereof, and
wherein the dietary fibers have a fermentation profile providing butyrate and propionate in a weight ratio of at least 1, preferably at least 1.1.

6. The at least one of C4 or C6 fatty acids and optionally C2 fatty acids thereof for use according claim 1, wherein the amount of C4 fatty acids is 0.2 mg to 12 mg per kilogram bodyweight per day and/or the amount of C6 fatty acids is 5 mg to 185 mg per kilogram bodyweight per day.

7. The at least one of C4 or C6 fatty acids and optionally C2 fatty acids for use according to claim 1, wherein the non-ketogenic ketogenic diet-booster composition is administered one to six times per day, preferably three to four times per day, or continuously, or wherein the non-ketogenic ketogenic diet-booster composition is administered continuously through a tube.

8. The at least one of C4 or C6 fatty acids and optionally C2 fatty acids for use according to claim 1, wherein the subject adhering to a ketogenic diet is suffering from a condition selected from the group of conditions consisting of
i) neurological diseases including epilepsy, stroke, traumatic brain injury, migraine;
ii) neurodegenerative diseases including Alzheimer's disease, Parkinson's disease and age-related mild cognitive impairment (MCI);
iii) neuromuscular diseases including amyotrophic lateral sclerosis, ageing-induced muscle inactivation, muscle wasting and sarcopenia, or elderly at risk of developing or suffering from frailty;
iv) psychiatric diseases including attention deficit hyperactivity disorder (ADHD) and autism spectrum disorders;
v) metabolic conditions including insulin resistance, type-1 and type-2 diabetes mellitus, Glucose transporter type 1 (Glut1) deficiency, Pyruvate dehydrogenase (PDH) deficiency, glycogen storage diseases and mitochondrial diseases;
vi) oncological disorders;
vii) cardiovascular diseases leading to heart failure.

9. The at least one of C4 or C6 fatty acids and optionally C2 fatty acids for use according to claim 1, wherein the subject adhering to a ketogenic diet is a subject suffering from epilepsy.

10. The at least one of C4 or C6 fatty acids and optionally C2 fatty acids for use according to claim 1, wherein top-level ketosis is increased.

11. The at least one of C4 or C6 fatty acids and optionally C2 fatty acids for use according claim 1, wherein the non-ketogenic ketogenic diet-booster composition comprises
(i) C4 fatty acids
(ii) C6 fatty acids
(iii) citric acid, and
(iv) nicotinamide riboside or nicotinamide, and optionally
(v) C2 fatty acids.

12. The at least one of C4 or C6 fatty acids and optionally C2 fatty acids for use according claim 1, wherein the non-ketogenic ketogenic diet-booster composition is administered at a dose of
(i) 0.2 mg to 12 mg per kilogram bodyweight per day C4 fatty acids
(ii) 6.5 mg to 163 mg per kilogram bodyweight per day C6 fatty acids
(iii) 0.011 gram to 0.39 gram per kilogram bodyweight per day citric acid, and
(iv) 0.6 mg to 14.3 mg per kilogram bodyweight per day nicotinamide riboside or equivalent dose range of nicotinamide, and optionally
(v) 0.5 mg to 30 mg per kilogram bodyweight per day C2 fatty acids.

13. A non-therapeutic method for
(i) enhancing ketosis and/or
(ii) preventing resistance to a ketogenic diet
in a healthy subject adhering to a ketogenic diet, comprising administering to the subject at least one of C4 or C6 fatty acids and optionally C2 fatty acids, wherein the one of C4 or C6 fatty acids and optionally C2 fatty acids are comprised in a non-ketogenic, ketogenic diet-booster composition, or the at least C2 or C4 fatty acids or derivatives thereof are provided in non-ketogenic amounts by a butyrogenic dietary fiber blend in a non-ketogenic, ketogenic diet-booster composition.

14. The non-therapeutic method according to claim 13 wherein the method is for promoting non-therapeutic weight loss and/or improving recovery after exercise and wherein optionally the promotion of non-therapeutic weight loss is in a subject having a BMI between 18 and 25, preferably between 20 and 25.

15. A non ketogenic, ketogenic diet-booster composition essentially consisting of
(i) 0.2 mg to 1.2 g C4 fatty acids;
(ii) 5 mg to 17 g C6 fatty acids;
(iii) 10 mg to 20 g citrate; and
(iv) 0.5 mg to 1.5 g nicotinamide riboside or nicotinamide wherein the amounts are based on the dry weight of the composition and wherein the amounts are per serving and wherein optionally the composition is in the form of a supplement.

16. (canceled)
